(19) United States
(12) Patent Application Publication
(10) Pub. No.: US 2016/0235866 A1
(43) Pub. Date: Aug. 18, 2016

(54) MRI CONTRAST AGENTS FOR CELL LABELING

(71) Applicants: THE GOVERNING COUNCIL OF THE UNIVERSITY OF TORONTO, Toronto (CA); THE HOSPITAL FOR SICK CHILDREN, Toronto (CA)

(72) Inventors: Xiao-An ZHANG, Toronto, Ontario (CA); Inga E. HAEDICKE, Toronto, Ontario (CA); Hai-Ling Margaret CHENG, Toronto, Ontario (CA)

(73) Assignees: The Governing Council of the University of Toronto, Toronto, ON (CA); The Hospital for Sick Children, Toronto, ON (CA)

(21) Appl. No.: 15/027,117

(22) PCT Filed: Oct. 6, 2014
(86) PCT No.: PCT/CA2014/050969

§ 371 Date: Apr. 4, 2016

Related U.S. Application Data
(60) Provisional application No. 62/034,344, filed on Aug. 7, 2014.
(60) Provisional application No. 61/887,279, filed on Oct. 4, 2013.

(51) Int. Cl. A61K 49/10; C07F 13/00; G01N 33/58; A61K 49/08

(52) U.S. Cl. CPC A61K 49/106; A61K 49/085; C07F 13/005; G01N 33/581

(57) ABSTRACT

Porphyrin compounds useful in the field of magnetic resonance imaging (MRI) as contrast agents. The compounds are relatively lipophilic porphyrins, include one or more enzyme-reactive functional groups, and are cell membrane permeable. Relatively lipophilic group(s) can be enzymatically released within a cell to produce a relatively hydrophilic porphyrin compound.

### FIELD OF THE INVENTION

The present invention relates to porphyrin compounds useful in the field of magnetic resonance imaging (MRI) as contrast agents, particularly as cellular contrast agents.

### BACKGROUND OF THE INVENTION

Cellular imaging has been critical in monitoring the function of endogenous or implanted cells under both physiological and pathological conditions. In recent years, there are increasing demands for new techniques to noninvasively monitor and track cells in vivo, as the field of cell transplantation, especially immune-cell and stem-cell therapy, is very rapidly expanding. In vivo cellular imaging technique is unique in its ability to visualize biological processes in real time at the cellular level and in an intact living subject.¹ Optical imaging (OI) has been at the forefront of the research field and is well-established with a large variety of fluorescent probes²⁻⁶ and fluorescent proteins⁷ available for labeling of specific cell structures, as well as observing dynamic cellular events such as gene expression.⁷ The technique itself is a highly sensitive imaging modality (nanomolar to femtomolar), with high spatial and temporal resolution.⁷ Unfortunately, OI is limited to applications with transparent subjects or superficial tissue depths due to light scattering and attenuation.⁸ Conventional medical imaging modalities including X-ray computed tomography (CT), ultrasound (US), positron emission tomography (PET), single photon emission computed tomography (SPECT) and magnetic resonance imaging (MRI) overcome this penetration limitation. Among these widely used techniques, CT has relatively low resolution and low sensitivity. PET and SPECT provide excellent sensitivity and information about cell function but are limited in terms of lack of anatomical information, restricted time window due to decay of radioactive nuclides, and exposure to ionizing radiation. US is safer than nuclear techniques but is limited by spatial resolution for cell tracking and also suffers from poor tissue contrast and a restricted field of view.⁹ In contrast, MRI is free of ionizing radiation, and can noninvasively offer detailed anatomic information with high spatial resolution (˜50 μm) and excellent soft tissue contrast while providing important functional information about physiology, such as blood flow.¹⁰ These major advantages of MRI have led to a plethora of research studies for tracking transplanted cells in vivo as well as several clinical investigations¹¹⁻¹⁴ following therapeutic cells in vivo with MRI.

Cell transplantation has shown promise for the treatment of a wide variety of medical conditions such as neurodegenerative disorders,¹⁵ diabetes,¹⁶ cancers,¹⁷⁻¹⁹ myocardial infarction,²⁰ and stroke.²¹ Non-invasive imaging of temporal changes in implanted cell distribution, viability and functional status are critical for further development of cell-based therapies. Selected groups of cells can only be visualized after intracellular uptake of a contrast agent. For most applications the cells are labeled with the MRI contrast agent (CA) in vitro prior to administration to the subject. This methodology circumvents the difficulties of targeting specific cells in vivo. The labeled cells can be tracked in vivo by MRI non-invasively and in the same subject over extended periods of time. In addition to the stem cells, research studies have shown MRI CA uptake for tracking a wide variety of other types of cells with therapeutic functions, such as lymphocytes, phagocytic cells and genetically engineered cells.^{22, 23, 1}

Conventional MRI typically relies upon ¹H-NMR signals from water and fat molecules, as they are the most abundant proton sources in vivo.²⁴ In addition to the proton density, there are additional parameters, including longitudinal (T₁) and transverse relaxation times (T₂), that determine MRI signal intensity and thereby image contrast. Differentiation of tissues on MR images results from varying proton densities and more predominantly, the inhomogeneity of T₁ and T₂ relaxation times of the corresponding tissues. By varying acquisition parameters such as pulse sequences, MRI can gather a variety of information about anatomy and function. In this way MRI is unique because a wide variety of pulse sequences are available for revealing anatomic details in organs.²⁵

For the differentiation of normal and diseased tissues however it is often necessary to use a contrast agent (CA) because the difference in native relaxation times are too small to detect. For the same reason, implanted cells need to labelled with a CA in order to be visualized by MRI. Therefore CAs are widely used to improve the sensitivity, contrast and specificity of MRI scans.²⁶ CAs function by shortening the T₁ and T₂ relaxation times of protons in the vicinity of the agent. They are broadly classified as T₁- or T₂-agents depending on their major influence on either the T₁ or T₂ relaxation times. Relaxivity (r) can be defined as the efficiency of a CA at reducing the T₁ (r₁) or T₂ (r₂) of nearby water protons.²⁷

T₁ contrast agents are coordination complexes of paramagnetic high-spin transition metals (Mn(II), Mn(III), Fe(III), etc.) and lanthanides (primarily Gd(III)) that result in a hyperintense or positive signal enhancement in T₁-weighted images. Enhanced relaxation of the surrounding water nuclei is induced through electron-nuclear spin-spin coupling from the unpaired electrons of the metal.²⁷ Most clinical T₁ CAs utilize Gd(III) because 7 unpaired f-electrons result in a large magnetic moment and the symmetrical ground state leads to a long electronic relaxation time thereby facilitating the relaxation of nearby hydrogen nuclei via spin coupling.²⁷ Due to heavy metal toxicity, Gd(III) must be chelated for in vivo applications. Typical examples of widely used clinical contrast agents are Magnevist™ or Gd(III)-diethylenetriaminepentaacetic acid (Gd-DTPA) and Dotaram™ or Gd(III)-1,4,7,10-tetraazacyclododecane-1,4,7,10-tetraacetic acid (Gd-DOTA). CAs are critical for disease diagnoses such as malignant tumors and are currently used in 40% of clinical MRI scans. These contrast agents were designed for administration into the circulatory system and as such are small hydrophilic compounds that facilitate rapid renal clearance to reduce toxicity.

T₂ agents lead to signal dephasing due to the magnetic field inhomogeneity induced in nearby water molecules that lead to hypointense or negative signal enhancements on T₂ and T₂* weighted images. T₂ agents are predominantly superparamagnetic particles such as iron oxide nanoparticles (e.g. magnetite Fe₃O₄ particles).²⁸

Currently the most widely used CAs for MRI-based cell imaging have been formulations of superparamagnetic iron oxide nanoparticles (SPIOs).^{28,29} There are several commercially available SPIOs³⁰ that have been used in an abundance of research studies as well as several clinical trials.¹ These nanoparticles have especially high relaxivities because the large magnetic moment of the particles disrupts the homogeneity of the magnetic field well beyond the size of the actual particle. These contrast agents are grouped according to their size into (a) ultrasmall superparamagnetic iron oxide nanoparticles (USPIO), with a diameter <20 nm, (b) SPIO, with a diameter=20-100 nm, (c) micron-sized paramagnetic iron oxide particles, (MIONs) that are ˜1 μm and larger. The particles are coated with organic polymers for increased stability, biocompatibility and potentially, for further chemical modification. The largest particles, MIONs exhibit the highest T₂ relaxivity and have been used in a seminal study by Koretsky et al that demonstrated that cells labeled with a single MION could be detected in live mouse embryos with MRI at 11.7 Tesla.³¹ The MIONs used in that study as well as several other formulations of SPIOs are commercially available³⁰ such as Ferridex I.V.™ (Advanced Magnetic Industries, Cambridge, Md., USA) consisting of ferumoxide particles that range in size from 120-180 nm, Sinerem™ (Guerbet, Villepinte, France) consisting of fermumoxtran-10 particles from 15-30 nm and Resovist™ (Bayer Schering, Pharma, Berlin, Germany) consisting of ferucarbotran particles 60 nm in size. An important example of a clinical study used FDA-approved SPIO ferumoxide Endorem™ (Guerbet, Villepinte, France) to successfully label therapeutic dendritic cells and monitor their migration in vivo, as cancer vaccines in melanoma patients.¹²

There are however, several major limitations associated with T₂-based CAs and IONs in particular. Since these particles are in general cell-impermeable they are limited to certain cell types that incorporate the particles through phagocytosis.

Therefore they have been primarily used to label phagocytic cells such as macrophages, microglia and immature dendritic cells.^{11,12} Alternatively, cell labeling requires the use of transfection agents³⁰ or physical perturbation of the cell membrane, such as electroporation to load the contrast agent into cells. The need for transfection agents (TAs) complicates the labeling procedure because each TA will require FDA approval. Furthermore it has been shown that certain SPIOs and TAs can form large particles that do not get endocytosed. Although viability and proliferation of labeled cells was unaffected, there have been no studies on the effects of the labeled cells on the host tissue. There are also limitations for determination of the cell volume and quantitative analysis of the signal because the large magnetic susceptibility of the particles induces strong image artifacts that affect an area that extends far beyond the volume of the labeled cells. This blooming affect is also detrimental for visualization of the fine delineation of the labeled cells from the surrounding tissue and leads to loss of anatomical information in the vicinity of the labeled cells. T₂ agents also suffer in terms of specificity since they generate signal voids on the image which are ambiguous because they can also be generated from blood vessels, air spaces, hemorrhages, tissue interfaces or other imaging artifacts associated with magnetic susceptibility.¹⁰ One method to overcome these limitations has been the development of pulse sequences that generate hyperintense contrast from SPIOs such as GRASP.³² However, these pulse sequences require specialized hardware and there are safety concerns with the use of multiple high-energy pulses. Furthermore, some of the normal anatomical background of the image may be lost. There is therefore a need to develop T₁ CAs with sufficient sensitivity to overcome these limitations.

There are currently three different types of positive CAs used for cell labeling. Traditional proton MRI contrast agents based on Gd(III)-complexes, Mn(II)-complexes and heteronuclear MRI/MRS of ¹⁹F-based compounds. Recently there have been a multitude of research studies and several clinical studies focused on ¹⁹F cellular MRI.³³ The contrast agents are composed of perfluorocarbon (PFC) nanoemulsions (with a single ¹⁹F NMR resonance) such as perfluoropolyether (PFPE) or perfluoro-15-crown-5-ether (PCE).^{9, 34} PFC's are chemically and biologically inert and their clearance behavior has been studied extensively in the past as surgical blood substitutes.^{9, 34 19}F MRI has no endogenous background signals and by using a reference compound, ¹⁹F NMR can easily quantify the cellular uptake and after in vivo administration can be used for a quantitative measure of the number of cells per voxel. In 2005, Eric T. Ahrens used PFPE to label and track dendritic cells in live mice.³³ In 2011 the contrast agent was approved by the FDA for a phase 1 clinical trial on dendritic cell based vaccines. The commercially available contrast agent has been used for labeling of a variety of phagocytic cells.

There are several factors preventing the wide spread use of these ¹⁹F-based contrast agents. Most importantly is the lower sensitivity of ¹⁹F MRI compared to traditional T₁ CAs since the local concentration of fluorinated molecules will always be much lower than proton containing molecules in vivo. Cells must therefore internalize significant numbers of PFC's and only a large number of cells in close proximity can be visualized (>200 cells/voxel).¹⁰ This is due to a lower signal to noise ratio (SNR) and also results in substantially longer image acquisition times. Heteronuclear MRI also requires extra hardware limiting the widespread use. Furthermore ¹H MRI is necessary for acquiring the anatomical information that gets superimposed with the ¹⁹F MRI for cell localization. A limitation shared by all of these contrast agents is the previously mentioned lack of cell permeability.

The conventional clinical MRI CAs are predominantly Gd-complexes. Since these agents were designed for intravascular injection and fast renal clearance, they are by necessity small and hydrophilic compounds. These CAs are inherently cell impermeable, since passive diffusion across the cell membrane generally requires hydrophobic characteristics. Therefore, most of these compounds require additional transfection agents or physical methods for cellular uptake.^{35, 36} Alternatively, these compounds can be systematically altered for the design and synthesis of a cell permeable contrast agent. Meade et al synthesized a series of Gd-DOTA derivatives with varying lengths of polyarginine oligomers.^{37, 38} Polyarginine oligomers are known for transporting charged and uncharged species across cellular membranes. All derivatives showed successful cellular labeling after only 1 h of incubation. The length of the oligomer was optimized for cellular uptake and T₁ relaxivity. The optimized compound Gd-DOTA-(arginine)₈ had the highest relaxivity (6.8 mM⁻¹ s⁻¹, 1.4 T, 37° C., pH=7.41) among the compounds tested. Cell permeability demonstrated by these compounds was an important first step to developing a contrast agent for labeling and tracking of non-phagocytic cells in vivo. However, cell permeability needs to be combined with a mechanism for cellular retention or the contrast agent will diffuse out of the cell. This approach was also demonstrated by Meade et al with the incorporation of a cleavable disulfide linkage between the Gd-DOTA moiety and the transduction domain.³⁹ Reduction of the disulfide linkage by glutathione (most abundant cytosolic reducing agent) was demonstrated and led to the expected increased intracellular retention from 15% retention after 24 h for Gd-DOTA-(arginine)₈ to 40% retention after 24 h for Gd-DOTA-SS-(arginine)₈.³⁹

Despite the progress made towards synthesis of cell permeable and trappable Gd-based CAs for cell labeling, these CAs have not found widespread use for a number of reasons. Low relaxivity of Gd-based CAs at fields above 1.0 T is an issue. Stronger magnetic fields lead to an increase in SNR and spatial resolution as well as decreasing the acquisition times, so cell labeling experiments are generally done with higher magnetic fields at 1.0 T or greater for animal models. Since T₁ CAs are already less sensitive on a molar basis than T₂ iron oxide based agents, it would be advantageous to obtain new T₁ contrast agents with substantial relaxivities even at high magnetic fields. Furthermore, intracellular biocompatibility has been raised as a concern for long term cell tracking experiments since these complexes are less stable at lower pH such as in lysosomes and free Gd(III) is a toxic heavy metal. As mentioned above, the ligands are necessary to avoid systemic toxicity and as intravascular agents, they are rapidly cleared in vivo by renal filtration.^{27, 40} However, cellular clearance mechanisms and long term effects brought about by cellular retention of Gd are not known.

A less toxic paramagnetic metal used in cell labeling is Mn(II), primarily as MnCl₂. Manganese is an endogenous micronutrient and Mn(II) ions are sufficiently taken up by a variety of cell types with different mechanisms, such as in neurons by voltage gated calcium channels. MnCl₂ has been applied for functional imaging of labeled cardiac muscle cells and neurons.⁴¹⁻⁴³ Labeling of T and B lymphocytes was also accomplished with MnCl₂.⁴² While these studies showed no change in viability or function of the labeled cells under the experimental conditions, free Mn(II) ions can be toxic at high concentrations.⁴⁴ In addition, most cells have mechanisms to maintain the endogenous micronutrients at healthy levels. The high concentration of intracellular Mn(II) cannot be maintained for long-term labeling. Finally, the relaxivity of Mn(II) is strongly field-dependent, the T₁ drastically decreases with increasing magnetic field strength but the T₂ is very high at high field. Therefore, positive T₁ effects can be cancelled out by negative T₂ effects especially at high intracellular concentration of Mn(II). Taken together, these drawbacks have limited the scope of MnCl₂ for cell labeling and long-term tracking in vivo.

Criteria for an ideal MRI contrast agent for cell labeling and in vivo tracking include a positive contrast mechanism for high specificity, sufficient relaxivity at high fields, low toxicity, optimum biocompatibility, high cell permeability for labeling various cell types, and a mechanism for increased cellular retention.

### SUMMARY OF THE INVENTION

The invention includes a metallized paramagnetic tetrapyrollic unit e.g., porphyrin or porphyrin analog linked to one or more relatively hydrophobic groups that can be cleaved in vivo to expose relatively hydrophilic group(s) attached to the tetrapyrollic unit. The relatively hydrophobic compound can thus be taken up by a cell i.e., cross a cell membrane into the cell interior. Hydrophobic group(s) are enzymatically cleaved in the cell interior to reveal corresponding hydrophilic group(s). The relatively hydrophilic product is less able to move across the cell membrane so is less prone to escape the cell interior.

A broad aspect of the invention is a compound in the form of a metallized paramagnetic tetrapyrollic contrast agent linked to a substituent, wherein the substituent is hydrolysable by an enzyme to form a relatively polar group. In this context, relatively polar means that the group formed after hydrolysis of the protected agent to form an unprotected, or unmasked, form of the agent is more polar, than in the protected group. The unprotected form is thus more hydrophilic than the protected form.

Compounds of the invention include paramagnetic porphyrin (or porphyrin analog)-containing compounds of formula (A), or a salt thereof:

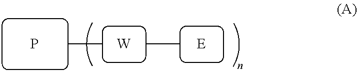

wherein the component denoted as P contains at least one or more paramagnetic porphyrins or porphyrin analogs, the component denoted as W contains at least one or more polar and hydrophilic groups, and the component denoted as E contains at least one or more enzyme-reactive protecting groups, which are used to fully or partially mask the polarity or hydrophilicity of W. Herein the combined components of W and E, is termed as “masked polar moiety” (W-E). The component denoted as P is linked to at least one or more (n≧1) masked polar moieties independently. The number of masked polar moieties is represented by the whole number n that can be 1, 2, 3, or 4 or greater. The component denoted as P and the masked polar moiety (W-E) are covalently linked. For compounds containing more than one masked polar moieties, each masked polar moiety can be different or the same. Examples of “paramagnetic porphyrin”, “polar and hydrophilic moiety” and “enzyme-reactive protecting group” are described further below.

In a preferred aspect, the hydrolysable substituent contains a hydrolysable covalent linkage selected from the group consisting of ester (—C(O)O—), ether (—O—), amide (—C(O)NH—), alkylamide (—C(O)NR—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage. Generally speaking, the linkage within the masked polarity moiety, W-E, is oriented such that after hydrolysis a heteroatom e.g., oxygen of an alcohol group, or of a carboxyl group is exposed in the unmasked, or deprotected form of the agent.

The linkage is typically cleavable (hydrolysable) by an esterase enzyme.

In embodiments, the invention is a paramagnetic compound having the formula (B):

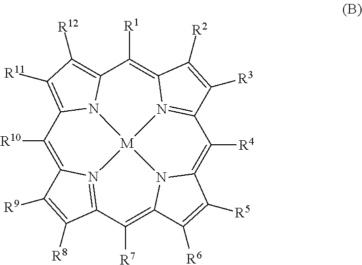

wherein M is a paramagnetic metal ion;
at least one of R¹ to R¹² is a said protected hydrolysable substituent, and each of the
remaining R¹ to R¹² is independently selected from the group consisting of: hydrogen;
   C₁-C₂₀ alkyl optionally substituted with up to 4 of any of hydroxyl, halogen, thiol, cyano, nitro, oxo (═O);
   C₃-C₂₀ cycloalkyl optionally substituted with up to 4 of any of hydroxyl, halogen, thiol, cyano, nitro, oxo (═O);
   C₃-C₂₀ heterocycloalkyl optionally substituted with up to 4 of any of hydroxyl, halogen, thiol, cyano, nitro, oxo (═O);
   C₁-C₂₀ alkenyl optionally substituted with up to 4 of any of hydroxyl, halogen, thiol, cyano, nitro, oxo (═O);
   C₃-C₂₀ cycloalkenyl optionally substituted with up to 4 of any of hydroxyl, halogen, thiol, cyano, nitro, oxo (═O);
   C₃-C₂₀ heterocycloalkenyl optionally substituted with up to 4 of any of hydroxyl, halogen, thiol, cyano, nitro, oxo (═O);
   C₆ to C₂₀ aryl optionally substituted with up to 4 of any of hydroxyl, halogen, thiol, cyano, nitro;
   C₃ to C₂₀ heteroaryl optionally substituted with up to 4 of any of hydroxyl, halogen, thiol, cyano, nitro;
   C₇ to C₂₀ arylalkyl optionally substituted with up to 4 of any of hydroxyl, halogen, thiol, cyano, nitro, oxo (═O);
   C₄ to C₂₀ heteroarylalkyl optionally substituted with up to 4 of any of hydroxyl, halogen, thiol, cyano, nitro, oxo (═O);
   C₂ to C₂₀ alkynyl optionally substituted with up to 4 of any of hydroxyl, halogen, thiol, cyano, nitro, oxo (═O);
   C₁ to C₂₀ heteroalkyl optionally substituted with up to 4 of any of hydroxyl, halogen, thiol, cyano, nitro, oxo (═O);
   C₂ to C₂₀ heteroalkenyl optionally substituted with up to 4 of any of hydroxyl, halogen, thiol, cyano, nitro, oxo (═O); and
   C₂ to C₂₀ heteroalkynyl optionally substituted with up to 4 of any of hydroxyl, halogen, thiol, cyano, nitro, oxo (═O).

An “alkyl” group indicates the radical obtained when one hydrogen atom is removed from a hydrocarbon. An alkyl group has 1 to 20, 1 to 12, such as 1 to 6, 1 to 5, 1 to 4, 1 to 3, 1 or 2 carbon atoms, or 1 carbon atom. The term includes the subclasses normal alkyl (n-alkyl), secondary and tertiary alkyl, such as methyl, ethyl, n-propyl, isopropyl, n-butyl, isobutyl, sec.-butyl, tert.-butyl, pentyl, isopentyl, hexyl and isohexyl.

A “cycloalkyl” group indicates a saturated cycloalkane radical having 3 to 20 carbon atoms, so can have 3 to 10 carbon atoms, in particular 3 to 8 carbon atoms, such as 3 to 6 carbon atoms, or 6 carbon atoms and includes fused monocyclic, bicyclic, polycyclic, fused, bridged, or spiro polycyclic ring structures, for example, cyclopropyl, cyclobutyl, cyclopentyl, cyclohexyl, or cycloheptyl.

A “heterocycloalkyl” denotes a cycloalkane radical as described above in which one or more CH₂ groups atoms e.g., 1, 2, 3 or 4 CH₂ groups are replaced by corresponding heteroatoms, 0 or S, or in which one or more CH groups are replaced by a corresponding heteroatom N, an example of which is piperazinyl.

An “alkenyl” group indicates an alkyl group in which 1, 2, 3, 4 or 5 unsaturations (double bonds) replace a corresponding number of —CHCH— groups, examples being ethenyl, propenyl, butenyl, pentenyl or hexenyl.

A “cycloalkenyl” group indicates mono-, di- tri- or tetraunsaturated non-aromatic cyclic hydrocarbon radicals such as containing 3 to 20 carbon atoms, including fused monocyclic, bicyclic, polycyclic, fused, bridged, or spiro polycyclic ring structures, and include groups containing 3 to 10 carbon atoms, such as 3, 4, 5 or 6 carbon atoms, e.g. cyclopropenyl, cyclobutenyl, cyclopentenyl, cyclohexenyl, cylcoheptenyl.

A “heterocycloalkenyl” indicates a cycloalkene radical (cycloalkenyl group) in which one or more CH₂ groups atoms e.g., 1, 2, 3 or 4 CH₂ groups are replaced by corresponding heteroatoms, O or S, or in which one or more CH groups are replaced by a corresponding heteroatom N, examples being dihydrofuranyl and 2,5-dihydro-1 H-pyrrolyl.

An “aryl” group is a radical of aromatic carbocyclic rings having 6 to 20 carbon atoms, such as 6 to 14 carbon atoms, or 6 to 10 carbon atoms, particularly 5- or 6-membered rings, that can be fused carbocyclic rings with at least one aromatic ring, such as phenyl, naphthyl, indenyl and indanyl.

A “heteroaryl” group is a radical containing at least one aromatic ring having 1 to 6 O, S and or N heteroatoms, and 1 to 20 carbon atoms, such as 1 to 5 heteroatoms and 1 to 10 carbon atoms, or 1 to 5 heteroatoms and 1 to 6 carbon atoms, in particular 5- or 6-membered rings with 1 to 4 heteroatoms, and can include fused bicyclic rings with 1 to 4 heteroatoms, and wherein at least one ring is aromatic, such as pyridyl, triazolyl, quinolyl, isoquinolyl, indolyl, tetrazolyl, thiazolyl, imidazolyl, pyrazolyl, oxazolyl, isoxazolyl, thienyl, pyrazinyl, isothiazolyl, benzimidazolyl and benzofuranyl.

“Arylalkyl” denotes an aryl radical covalently joined to an alkyl group such as a benzyl group.

A “heteroarylalkyl” group indicates a heteroaryl radical covalently joined to an alkyl group.

An “alkynyl” group is a hydrocarbon radical having 1 to 5 triple C—C bonds —C≡C—) and 2 to 20 carbon atoms, typically having 2 to 10 carbon atoms, or 2 to 6 carbon atoms, such as 2 to 4 carbon atoms, examples being ethynyl, propynyl, butynyl, pentynyl or hexynyl.

“Heteroalkyl, heteroalkenyl, heteroalkynyl” refer to alkyl, alkenyl and alkynyl groups, respectively, in which one or more of the carbon atoms (and any associated hydrogen atoms) are each independently replaced with the same or different heteroatoms O, S or N.

“Halogen” indicates a substituent from the seventh main group of the periodic table: fluoro, chloro, bromo and iodo.

The term “haloalkyl” indicates an alkyl group substituted with one or more halogen atoms as defined above, e.g. difluoromethyl. An alkyl optionally substituted with halogen is a haloalkyl when so substituted.

In general, an optional substitution with specified groups, radicals or moieties means that the subsequently described substitution may or may not occur, so that the description includes instances where the circumstance occurs and instances where it does not. An atom with unsatisfied valence(s) is assumed to have the hydrogen atom(s) to satisfy the valences.

“Phosphate” refers to a radical —OP(O)(OR′)(OR″) where R′ and R″ are each independently hydrogen, alkyl, cycloalkyl, cycloheteroalkyl, aryl, arylalkyl, heteroalkyl, heteroaryl or heteroarylalkyl.

“Sulfonate” refers to a radical —S(O)(O)OR′, where R′ is hydrogen, alkyl, cycloalkyl, cycloheteroalkyl, aryl, arylalkyl, heteroalkyl, heteroaryl or heteroarylalkyl.

“Carboxy” or “carboxyl” means the radical —C(O)OH. It is also to be understood that terms encompass such groups whether or not in ionized form as part of the compound, so cover salts such as sodium carboxylate (—CO₂⁻Na⁺), etc.

The term “hydroxyalkyl” denotes an alkyl group substituted with one or more hydroxyl groups such as hydroxymethyl, hydroxyethyl, hydroxypropyl.

An “alkoxy” group indicates a radical of the formula —OR′ in which R′ is alkyl such as methoxy, ethoxy, n-propoxy, isopropoxy, butoxy, etc.

The term “alkoxycarbonyl” indicates a radical of the formula —C(O)—O—R′ in which R′ is alkyl, such as methoxycarbonyl, ethoxycarbonyl, n-propoxycarbonyl, iso-propoxycarbonyl, etc.

The term “alkylcarbonyl” indicates a radical of the formula —C(O)—R′ in which R′ is alkyl, such as acetyl.

A “heterocyclic ring” includes heteroaryl, heterocycloalkyl and heterocylcoalkenyl and further includes annelated ring systems with each other or with cyclic hydrocarbons.

The term “pharmaceutically acceptable salt” indicates salts formed by reacting a compound of formula (A) with a suitable inorganic or organic acid, such as hydrochloric, hydrobromic, hydroiodic, sulfuric, nitric, phosphoric, formic, acetic, 2,2-dichloroaetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula (A) may also be prepared by reaction with a suitable base such as sodium hydroxide, potassium hydroxide, magnesium hydroxide, calcium hydroxide, silver hydroxide, ammonia or the like, or suitable non-toxic amines, such as lower alkylamines, for example triethylamine, hydroxy-lower alkylamines, for example 2-hydroxyethylamine, bis-(2-hydroxyethyl)-amine, glucamine, N-Methylglucamine cycloalkylamines, for example dicyclohexylamine, or benzylamines, for example N,N′-dibenzylethylenediamine, and dibenzylamine, or L-arginine or L-lysine. Salts obtained by reaction with a suitable base include, but are not limited to sodium salts, choline salts, 2-(dimethylamino)-ethanol salts, 4-(2-hydroxyethyl)-morpholin salts, L-lysine salts, N-(2-hydroxyethyl)-pyrrolidine salts, ethanolamine salts, potassium salts, tetrabutylammonium salts, benzyltrimethylammonium salts, cetyltrimethylammonium salts, tetramethylammonium salts, tetrapropylammonium salts, tris(hydroxymethyl)amino-methane salts, N-methyl-D-glucamine salts, silver salts, benzethonium salts, and triethanolamine salts.

According to preferred aspects, the at least one of R¹ to R¹² is selected from the group consisting of:

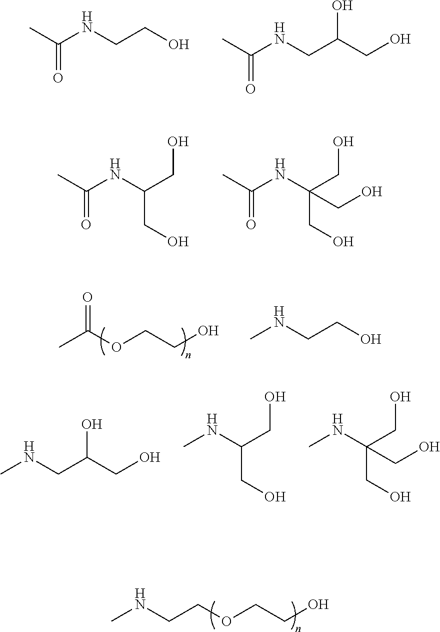

for n=1 to 20,
wherein at least one of the hydroxyl groups is replaced by a linkage covalently linked to a protecting group selected from the group consisting of: ester (—C(O)O—), ether (—O—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage.

The at least one of R¹ to R¹² can be selected from the group consisting of:

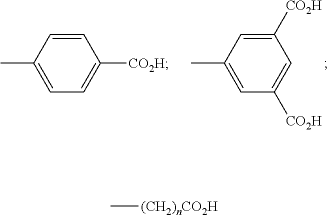

wherein the hydrogen atom of least one carboxyl group is substituted by a C₁-C₂₀ alkyl in which each hydrogen atom is optionally substituted with a halogen atom.

In another aspect, the at least one of R¹ to R¹² is:

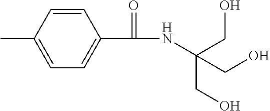 [SMILES: Cc1ccc(C(=O)NC(CO)(CO)CO)cc1]

wherein at least one of the hydroxyl groups is replaced by a linkage covalently linked to a protecting group selected from the group consisting of: ester (—C(O)O—), ether (—O—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage.

Particular a leaving groups (protecting groups) that can be covalently bonded to a said linkage are “L” in which:

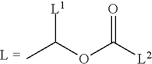

and L¹ and L² are independently selected from H and (C1-C20) alkyl, in particular:

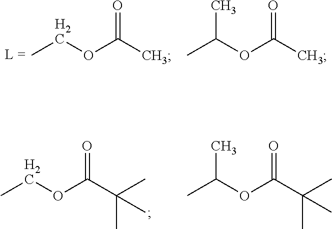

The number of masked polar moieties, n, as shown in formula (A), can be one or larger than one. For a compound containing a single porphyrin ring, n can be any of 1, 2, 3, 4, 5, 6, 7, 8, 9, 10, 11 or 12. Selected examples are shown below, with the number of masked polar moieties varied from 1 to 4:

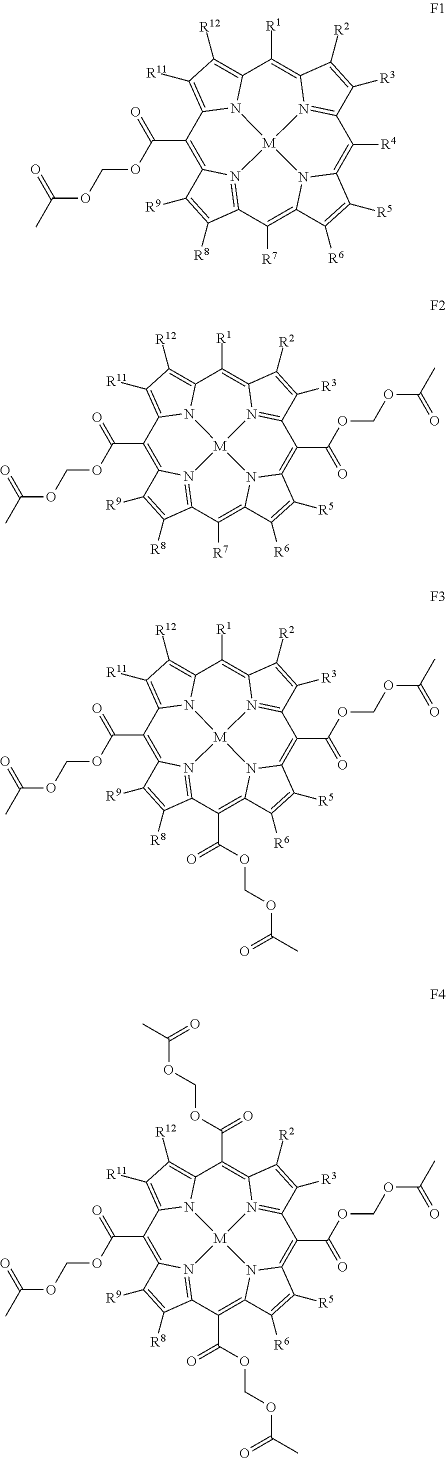

for R¹ to R¹² being independently defined as above for (B).

Other particular compounds are those in which R¹, R⁴, R⁷ and R¹¹ are independently selected from H and the following:

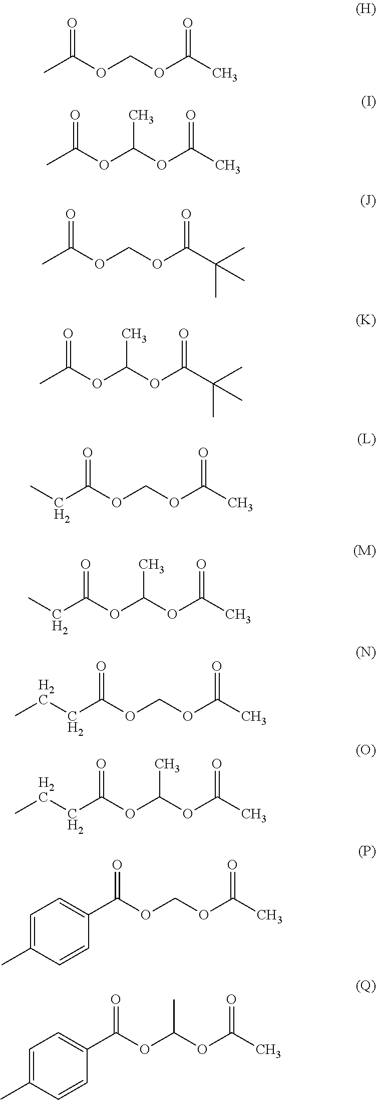

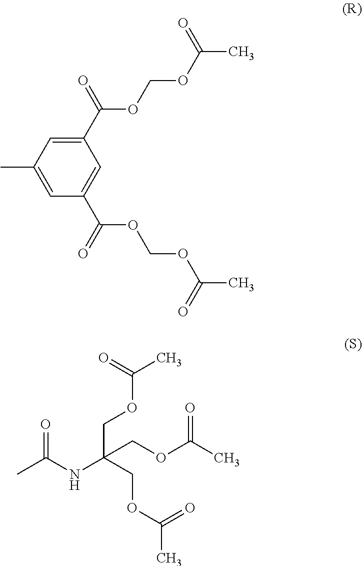

The position of the masked polar moieties on the porphyrin can be chosen at any positions among R¹-R¹² in formula (B). While the examples shown above are all on meso-positions (positions R¹, R⁴, R⁷ and R¹⁰), due to relative ease of synthesis, in other embodiments, the masked polar moieties can be located at beta-positions (positions R², R³, R⁵, R⁶, R⁸, R⁹, R¹¹, and R¹²). In these cases, formulas (L), (M), (N) and (O) are preferred choices as masked polar moieties. Selected examples are:

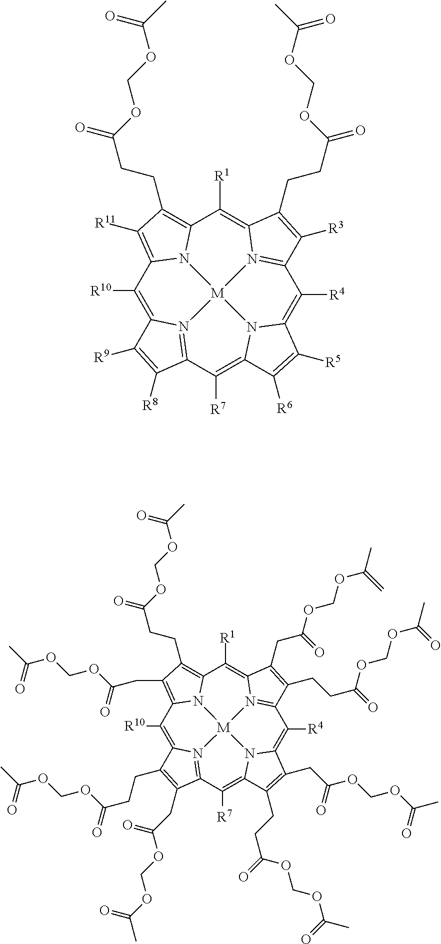

Two Mn-porphyrins among the preferred embodiments are:

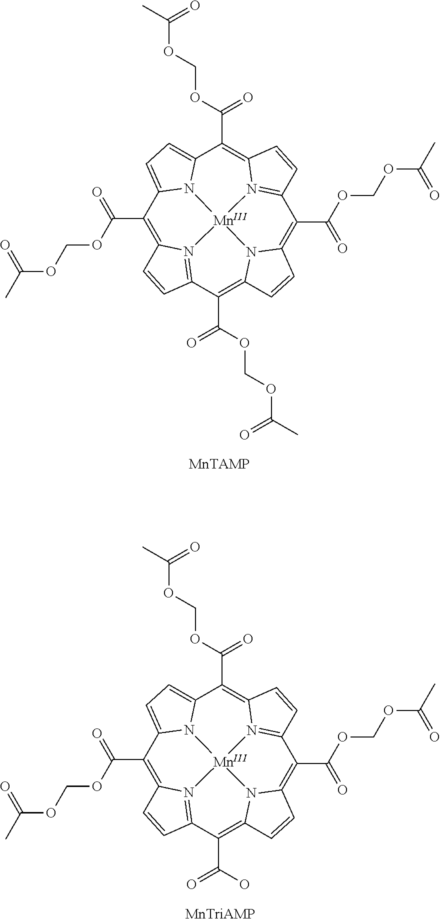

A compound of the invention can include multiple protected tetrapyrollic units linked to each other in the form of a protected metallized paramagnetic contrast agent. In embodiments, first and second porphyrin rings have the structure of respective formulas (P) and (P′):

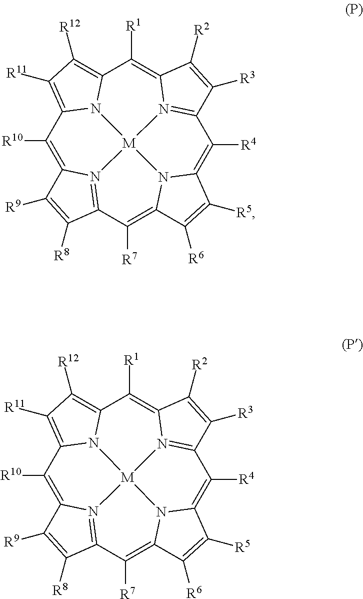

one of R¹ to R¹² of each of (P) and (P′) is independently a link to the other of (P) and (P′), the link being a covalent bond or the diradical of biphenyl (—C₆H₄—C₆H₄—), there being one, two, three or four porphyrin rings (P) linked to (P′);
at least one of the remaining R¹ to R¹² of each said porphyrin ring (P) is a hydrophilic substituent having a protecting group covalently linked thereto, the covalent linkage of the protecting group being hydrolysable by an enzyme under physiologic conditions to produce an unprotected agent that is relatively hydrophilic with respect to the protected agent;
optionally, each said (P) linked to (P′) is linked to another porphyrin ring (P); and each remaining R¹ to R¹² is as defined above for formula (B). By “physiological pH” or “physiological conditions” is meant water at a pH of about 7.5 and about 37° C., and an ioinic strength of about 150 mM. Acidic groups, such as carboxyl groups that exist mainly as negatively charged groups under physiological conditions.

For oligomeric forms with more than two porphyrin units, the number of porphyrin units and the connections between porphyrins may have significant influence on the geometry and size of the molecules. Preferred examples include:

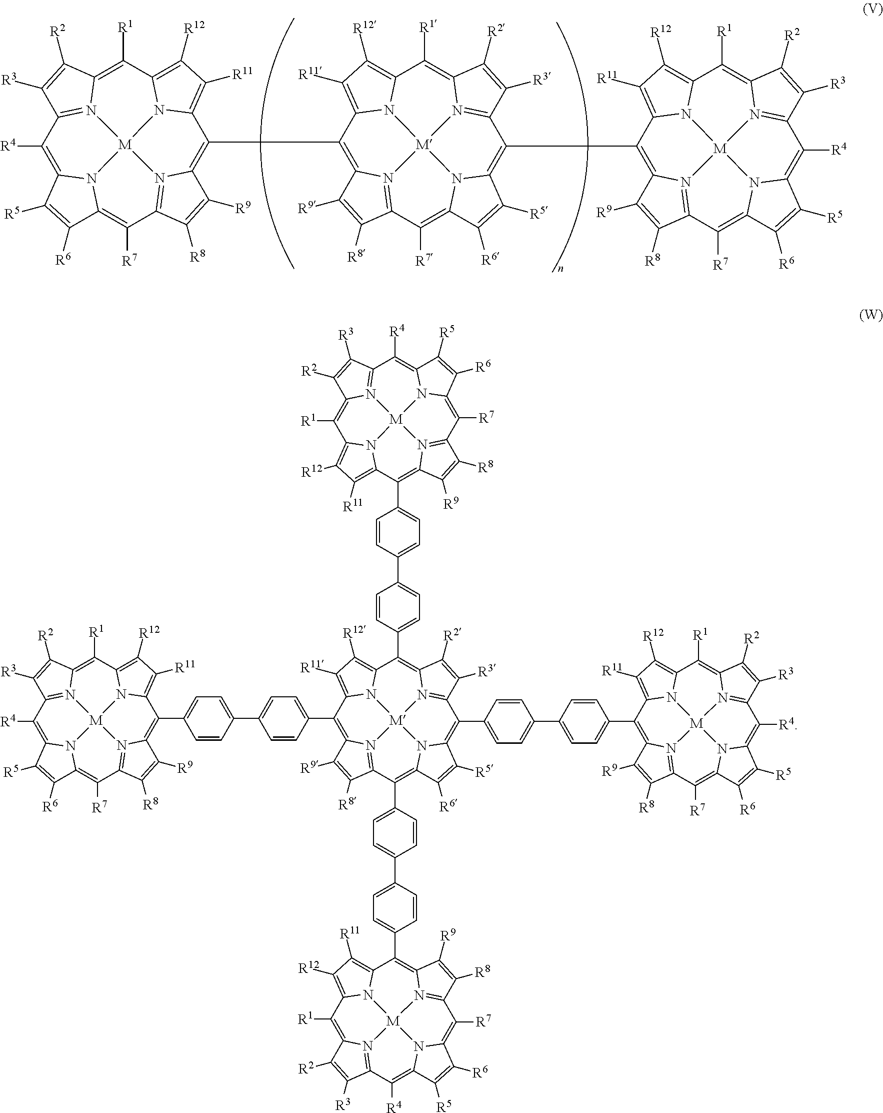

Paramagnetic metals of the metallized (metalated) compounds of the invention include Mn(II), Mn(III), Fe(II), Fe(III), Gd(III), Cu(I), Cu(II), Ni(II), Ni(I) and Ni(III). Advantageously, the ion can be Mn(II) and Mn(III), also referred to as Mn2+ and Mn3+, respectively, due to its relatively low toxicity. Mn(III) is preferred among the two oxidation states, due to the higher stability in porphyrin. In compounds containing multiple porphyrin rings, the metal in each ring of a said compound may be the same or different.

Preferably, paramagnetic porphyrins of the invention are free of toxic Gd, and exhibit high relaxivity at high magnetic fields in living cells without significant influence on cell viability, and can therefore be widely applied as cell-trappable MRI contrast agents for labeling cells and tracking cells in vivo.

Preferred agents of the invention are T₁ CAs based on manganese porphyrins (MnPs). It is expected that Gd-free porphyrins of the invention can be optimized to have high T₁ relaxivity at high magnetic field and high biocompatibility for in vivo applications. Through further rational structural modifications, the polar, hydrophilic and cell-impermeable MnP precursors can be transformed into lipophilic and highly-permeable compounds that can be rapidly taken up by cells during the labeling process. Once inside the cell, specific intracellular enzymes e.g., esterases, will readily convert the lipophilic MnPs back into the polar and cell membrane impermeable form, which will be retained intracellularly, thus facilitating long-term cell-labeling and tracking.

The mechanism of cell-permeable and trappable paramagnetic porphyrins for cell-labelling is schematically shown in Scheme 1. The general strategy is to include one (or more) polar and hydrophilic moiety(ies), denoted as W, and covalently linked to the paramagnetic porphyrin structure, P. The polarity and/or hydrophilicity of the polar moiety W is masked with an enzyme-reactive protection group, E, to make the paramagnetic porphyrin lipophilic and thereby make it relatively membrane-permeable in comparison to the porphyrin bearing only the W group(s). Once entering the cell, the enzyme-reactive protection group E can be cleaved by intracellular enzymes, releasing the polar, water-soluble form, P-(W)ₙ, which is much less membrane permeable than the protected or masked compound. This facilitates retention of the porphyrin in the cell interior for long-term labeling.

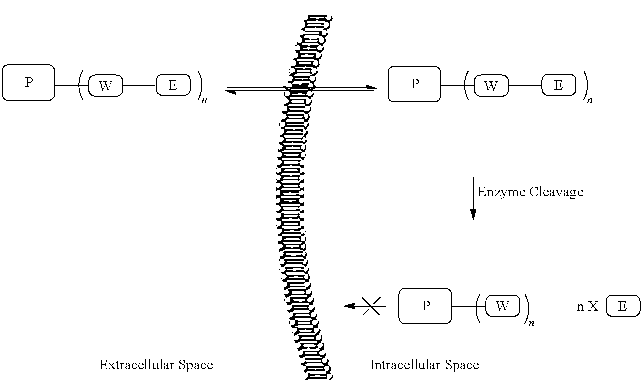

Scheme 1. Mechanism of cell-permeable and trappable paramagnetic porphyrins as MRI contrast agents for cell-labeling.

When the polar and hydrophilic group is an acidic group or the corresponding deprotonated anionic form described above, the preferred enzyme-reactive protecting group is in an ester form, which can be cleaved by esterase, and the corresponding masked polar moiety is of a general formula (E):

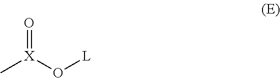

wherein L is an alkyl group, such as ethyl, methyl and tert-butyl groups, or derivatives of alkyl groups, which can be cleaved by esterases to release the polar and hydrophilic acidic group, in it's neutral (C) or deprotonated form (D).

Here, the derivatives of alkyl groups refer to a carbon-containing group with a heteroatom embedded in the group, which is connected to the hydroxyl oxygen of the polar and hydrophilic group described above, through a carbon atom. A preferable type of derivative of alkyl groups is of formula (F):

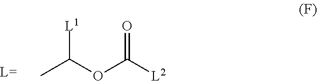

wherein L¹ and L² can be hydrogen or alkyl groups, independently.

This class of masked polar moieties can react with esterases, which cleave the L² acetyl bond to give a hemiacetal intermediate, which is instantly hydrolyzed in aqueous media to release the polar and hydrophilic group, as shown below:

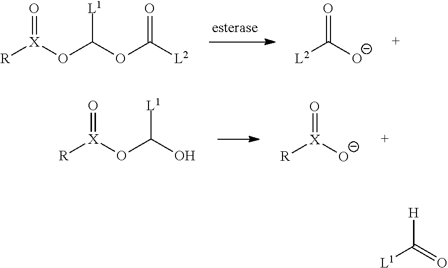

Some preferred examples in this class are shown below:

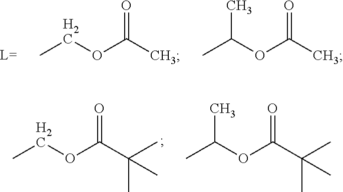

When L¹=hydrogen and L²=methyl group, the L is an acetoxymethyl (AM) group.

When a polar and hydrophilic group is a hydroxyl group or a moiety containing multi hydroxyl groups described above, the preferred enzyme-reactive protecting group is in an ester form, which can be cleaved by an esterase, and the corresponding masked polar moiety is of a general formula (G):

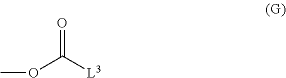

wherein L³ can be hydrogen or an alkyl group, preferably methyl and tert-butyl groups, or derivatives of alkyl groups described in (i).

When the polar and hydrophilic moiety contains more than one polar and hydrophilic group, it is possible that not all polar and hydrophilic groups are protected with enzyme-reactive protecting groups. Among those protected, different or the same protecting groups may be used.

In embodiments, the number, type, position and composition of masked polar moiety (W-E) described above can all be varied, as exemplified further below.

One or more of the substituents among R¹-R¹² in formula (B) can be the masked polar moieties, chosen preferentially from, but not limited to, foregoing formulas (H) to (S) as listed above. Selected examples are shown below, as of formula F2, F4-F33:

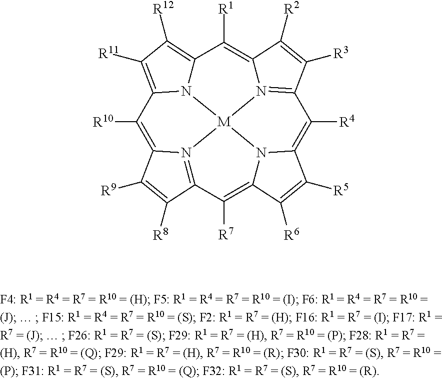

Formulas F4-F15 all have four of the same masked polar moieties at the meso-positions on porphyrin (positions R¹, R⁴, R⁷ and R¹⁰); formulas F2, F16-F26 all have two masked polar moieties at the meso-positions, para to each other, on the porphyrin (positions Wand R⁷, or positions Wand R¹⁰); formulas F29-F32 have four masked polar moieties, in two different types, at the meso-positions on porphyrin, of which the masked polar moieties para to each other (positions Wand R⁷; positions R⁴ and R¹⁰) are the same type.

In the embodiments, the paramagnetic porphyrin can be of monomeric form, and can also be dimeric, oligomeric or polymeric forms, wherein the substituents among R¹-R¹² in formula (B) contain one or more porphyrins. The dimeric, oligomeric and polymeric porphyrins can effectively increase the number of paramagnetic metal ions per molecule and slow down the rotational diffusion rate of the molecule, increasing the MRI T₁ relaxivity, which increases the sensitivity for MR cell imaging. In such embodiments, the linkers between porphyrin units, the size, geometry and paramagnetic species may vary, as exemplified below:

As illustrated above, neighboring porphyrin units can be linked to each directly through a covalent bond ((T)), or via a covalent linker ((U) below).

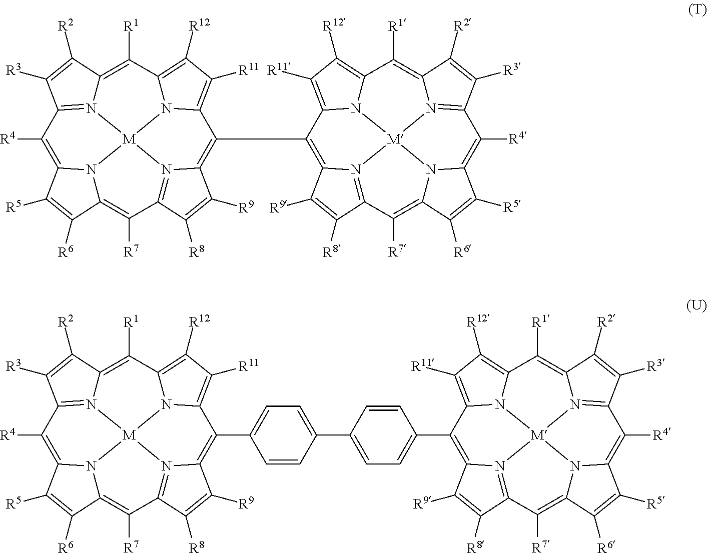

(i) For dimeric, oligomeric and polymeric porphyrins, whether they complex with a metal ion, and the choice of metal ion species in each porphyrin are independent. It is possible for there to be more than one type of metal ion, paramagnetic or diamagnetic to be incorporated as part of a compound. At least one porphyrin ring of a polyporphyrin compound of the invention is metalated with a paramagnetic ion, but it is thought preferable that all porphyrin rings of a polyporphyrin compound be metalated for use as an MRI contrast agents.

The invention includes a pharmaceutical formulation comprising a compound or salt thereof, as described herein, and a pharmaceutically acceptable carrier, wherein the formulation is suitable for administration as an imaging enhancing agent and the contrast agent is present in an amount sufficient to enhance a magnetic resonance image.

An embodiment of the invention is a method of generating an image of cells of a subject, the method comprising administering a compound or salt thereof, and generating an image of cells to which said compound has been distributed.

An embodiment includes a method of enhancing an image of a cell, the method comprising steps of:
exposing the cell to a contrast agent comprising a porphyrin ring covalently linked to a hydrophobic group by an ester linkage;
waiting a sufficient time permit the contrast agent to migrate across the cell membrane into the interior of the cell and for an esterase of the cell to cleave the hydrophobic group from the ring to generate relatively hydrophilic contrast agent in the interior of the cell; and
generating an image of the cell.

The contrast agent can further include a relatively hydrophilic group linking the porphyrin ring and said ester linkage such that the hydrophilicity of the agent increases upon the cleavage by the esterase.

The cell can be a stem cell, immune cell, blood cell, neuron, or beta cell.

The image is preferably an MRI.

An embodiment of the invention is a method of imaging a tumor and surrounding tissue in a subject comprising administering to the subject a composition comprising a compound or salt thereof, as described herein, and imaging the tumor and surrounding tissue in said subject.

The invention includes a composition containing a compound or salt thereof, as described herein, and a pharmaceutically acceptable carrier, excipient or diluent, suitable for administration to a subject.

Known methods for administering diagnostics can be used to administer CAs. For example, fluids that include pharmaceutically and physiologically acceptable fluids, including water, physiological saline, balanced salt solutions, buffers, aqueous dextrose, glycerol or the like as a vehicle, can be administered by any method used by those skilled in the art. These solutions are typically sterile and generally free of undesirable matter. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected. The invention further provides formulations comprising CA and a pharmaceutically acceptable excipient, wherein the CA is formed according to any of the embodiments described herein, and wherein the formulation is suitable for administration as an imaging enhancing agent and the CA is present in an amount sufficient to enhance an MRI image. These agents can be administered by any means in any appropriate formulation. Detergents can also be used to stabilize the composition or the increase or decrease the absorption of the composition. Other physiologically acceptable compounds include wetting agents, emulsifying agents, dispersing agents or preservatives that are particularly useful for preventing the growth or action of microorganisms. One skilled in the art would appreciate that the choice of acceptable carrier, including a physiologically acceptable compound depends, e.g. on the route of administration and on the particular physio-chemical characteristics of any co-administered agent.

Methods of introduction include, but are not limited to, intradermal, intramuscular, intraperitoneal, intravenous, subcutaneous, intranasal, rectal, vaginal, and oral routes. A CA composition may be administered by any convenient route, for example by infusion or bolus injection, by absorption through epithelial or mucocutaneous linings e.g., oral mucosa, vaginal, rectal and intestinal mucosa, etc., and may be administered together with other biologically active agents. Administration can be systemic or local. In addition, a CA composition may be introduced into the central nervous system by any suitable route, including intraventricular and intrathecal injection; intraventricular injection may be facilitated by an intraventricular catheter, for example, attached to a reservoir, such as an Ommaya reservoir. A CA composition can be delivered by any means known in the art systematically e.g., intra-venously, regionally or locally e.g. intra- or peri-tumoral or intra-cystic injection, e.g. to image bladder cancer by e.g., intra-arterial, intra-tumoral, intra-venous, parenteral, intra-pneural cavity, etc. For example, intra-arterial injections can be used to have a regional effect e.g. to focus on a specific organ (e.g. brain, liver, spleen, lungs). For example intra-hepatic artery injection or intra-carotid artery injection may be used. If it is decided to deliver the preparation to the brain, it can be injected into a carotid artery or an artery of the carotid system of arteries e.g., ocipital artery, auricular artery, temporal artery, cerebral artery, maxillary artery, etc. The present invention includes pharmaceutical compositions which include a CA alone or with a pharmaceutically acceptable carrier.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

### BRIEF DESCRIPTION OF DRAWINGS

Embodiments will now be described, by way of example only, reference being made to the accompanying drawings, in which:

FIG. 1 is an MRI of cell pellets: control, DMSO only, 1-MnTCP and 2-MnTriAMP.

### DETAILED DESCRIPTION

Certain MnPs can reach unusual high relaxivity, especially at high magnetic fields of clinical scanners. In 1984, Chen et al first reported the relaxivity of MnTPPS as ˜12 mM⁻¹s⁻¹ at 0.47 T (20 MHz for ¹H Larmor frequency), 37° C.⁴⁵ A measurement of the T₁ nuclear magnetic resonance dispersion (NMRD) profile of MnTPPS was subsequently reported by Konieg et al.⁴⁶ The NMRD profile (field-dependent relaxivity) for MnTPPS is unique among the known small molecule T₁ agents, because the relaxivity increases with magnetic fields above 1 MHz. The opposite trend has been shown for most small Gd-based as well as Mn(II)-based CAs where the NMRD profile decreases with increasing field strength. These results were surprising because Gd(III) has seven unpaired electrons (S=7/2) and Mn(II) has five unpaired electrons (S=5/2) compared to Mn(III) with only four unpaired electrons (S=4/2). Therefore, MnTPPS was described as having “anomalous high relaxivity”.⁴⁶ This uniquely high relaxivity at high fields is beneficial for the development of new CAs for MR cell imaging.

MnPs are highly stable against Mn-dissociation and free Mn is less toxic than Gd. Porphyrins have a fully conjugated 7-system leading to a conformationally rigid macrocycle with a predefined, metal binding pocket compatible for Mn(III) ions leading to a thermodynamically and kinetically stable complex. This also results in the correct geometry and molecular orbital energy levels for Mn (d) to ligand (π*) back-bonding, further increasing the bond strength as evident from spectroscopic studies of MnPs, thereby further reducing the likelihood of metal leakage.²⁷ While Mn, an endogenous micronutrient, is less toxic than Gd(III), there are still concerns about high concentrations of free Mn(II) in vivo, and a highly stable chelate would be desirable to eliminate any concerns about toxicity especially for longitudinal tracking of labeled cells in vivo.

MnPs are amenable to structural modifications. While the Mn is bound in the middle of the porphyrin core, different functional groups can be introduced at peripheral sites on the porphyrin ring, including meso- and beta-positions, without significantly influencing the Mn-binding. These possibilities for structural modifications allow systematic engineering of the molecular parameters of MnPs, including lipophilicity, water-solubility, charge, size, etc, thereby controlling the cell-permeability, intracellular and in vivo distribution, and biocompatibility. In the current invention, we introduced enzyme-reactive groups attached on the porphyrin ring, leading to cell-permeable and trappable MnPs.

### SYNTHESIS

In general, porphyrins can be synthesized from pyrrole and a chosen aldehyde via the Lindsey method.⁴⁷ Functional group modifications, manganese insertion and addition of esterase labile bonds lead to cell permeable and trappable MRI contrast agents. Alternatively, monomeric porphyrin building blocks can be subjected to dimerization, oligomerization or polymerization followed by functional group modification manganese insertion and installation of esterase labile groups. Sequence of these reactions steps may vary depending on the structure of the substrates.

### Synthesis of Porphyrin Building Blocks

Porphyrin monomers can be synthesized by the Lindsey reaction. Different aldehydes condensed with pyrrole followed by oxidation give access to a variety of symmetric (R₁=R₂=R₃=R₄) or non-symmetric (at least two R groups are different) porphyrins, as exemplified in Scheme 2. The symmetric tetrakis(ethoxycarbonyl)porphyrin (R₁=R₂=R₃=R₄=—COOEt), 1, was prepared using this method.

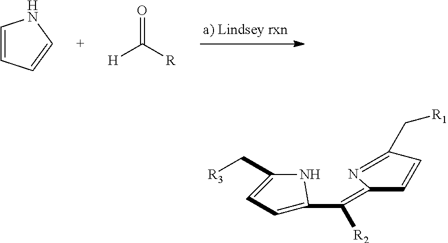

Scheme 2: General method for synthesis of symmetric or non-symmetric porphyrin monomers a) 1. BF₃.OEt₂, DCM; 2. DDQ.

### Mn Insertion and Functional Group Transformation

The porphyrin monomers were subjected to manganese insertion, to give several contrast agents. All other MnPs were deprotected and a varying number of acetoxymethyl ester groups were installed at the meso positions for cell permeability and hydrolysis by intracellular esterase.

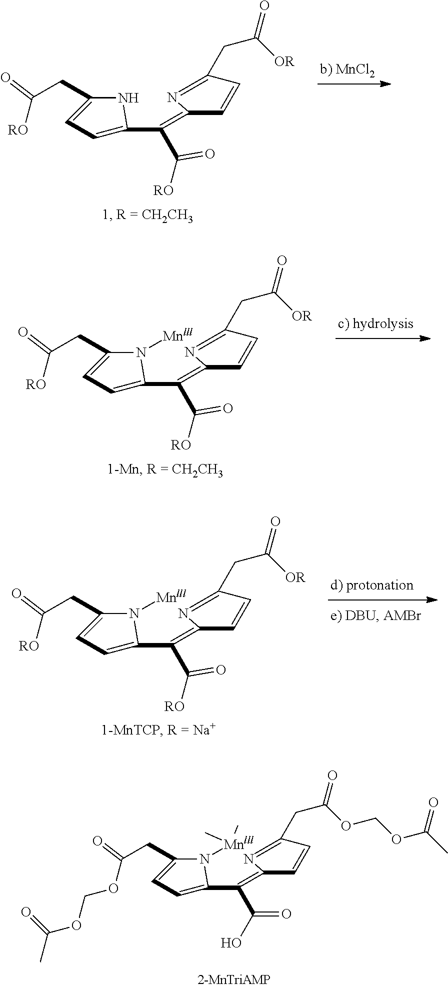

Scheme 3: Examples of Mn-insertion, deprotection and installation of esterase labile groups (in this example acetoxymethyl ester groups). b) Mn insertion: MnCl₂, DMF, reflux c) 2 M NaOH, THF, EtOH, reflux, 12 h d) 1 M HCl_{(aq)} e) DBU, AMBr, 55° C., 30 hours.

### Examples of Experimental Procedures

All reagents and solvents were of commercial reagent grade and were used without further purification except where noted. All reactions were carried out with oven dried glassware, anhydrous solvents and under argon atmosphere unless stated otherwise. Column Chromatography was carried out using Caledon Silica Gel 60; 50-200 microns 70-300 mesh, or using Sephadex™ LH-20 with dry bead size of 18-111 □m from GE Health Care. Dialysis was performed with spectrumlabs Float-A-Lyzer™ G2 500 MWCO. Cation ion exchange was performed using an Amberlite® IR120, H resin. Phosphate Buffer Saline was purchased from Sigma® Life Science, sterile filtered and endotoxin tested. The cell line was obtained from ATCC (American Tissue Culture Collection Manassas, Va., USA). 1640-RPMI medium was purchased from Sigma-Aldrich, trypsin EDTA was purchased from Gibco (Carlsbad, Calif., USA). All the spectroscopy data for structural characterizations were obtained using the research facilities at University of Toronto Scarborough campus (TRACES center) or at St. George campus (Chemistry Department). NMR spectra were recorded on Brucker-500 MHz or Varian Unity 500 MHz spectrometer. UV-vis spectra were recorded on an Agilent 8453 UV-Visible Spectroscopy System. Infrared spectra were recorded on a Bruker Alpha FT-IR Spectrometer. High resolution mass spectra were obtained from an ABI/Sciex Qstar mass spectrometer (ESI).

Synthesis of 5,10,15,20-tetrakis(ethoxycarbonyl)porphyrin, 1 was performed with a modification of a literature method.⁴⁸

Ethyl glyoxalate (50% in toluene, 1.88 ml, 9.4 mmol) in dichloromethane and pyrrole (0.65 ml, 9.4 mmol) were stirred at room temperature, in the dark and under an argon atmosphere. After 10 minutes BF₃.OEt₂ (42 ml, 3.10 mmol) was added drop wise. The reaction was stirred at room temperature for 1.25 hours followed by the addition of DDQ (1.5999 g, 7.05 mmol). After a stirring period of 2.25 hours NEt₃ (0.43 ml, 3.06 mmol) was added via syringe and the reaction mixture was concentrated on a rotary evaporator. The crude solution was suction filtered over sealite using DCM as an elution solvent. The solution was concentrated on a rotary evaporator. Purification by column chromatography (DCM) on silica gel gave 169.2 mg (12%) of compound 1 as a black-purple solid. ¹H NMR (CDCl₃) 9.52 (8H, s, por-β), 5.11 (8H, q, J=7.2), 1.81 (12H, t, J=7.2), −3.33 (2H, s, NH). UV-vis (DCM) λₘₐₓ=409 nm.

The synthesis of [5,10,15,20-tetrakis(ethoxycarbonyl)porphyrinato]manganese(III) chloride 1-Mn was performed according to a literature method.⁴⁸

Compound 1 (17.8 mg, 29.7 μmol) was dissolved in 2 ml of DMF. MnCl₂.4H₂O (17.7 mg, 89.2 μmol) was added and the reaction was refluxed open to air for 5 hours. The reaction was stirred at room temperature open to air for a further 11.5 hours. Distillation of DMF resulted in a black-purple solid. Purification by stepped gradient column chromatography (eluting with DCM to 7% MeOH in DCM) on silica gel gave 16.5 mg (85%) of compound 1-Mn as a black-purple solid. ESI MS found m/z=651.1 [M+], calcd for C₃₂H₂₈MnN₄O₈⁺, m/z=651.1. UV-vis (MeOH) λ_{abs}=328, 366, 387, 413, 456, 552 nm.

Synthesis of [5,10,15,20-tetrakis(carboxy)porphyrinato]manganese(III) chloride 1-Mn TCP was carried out as described in Canadian Patent Application No. 2,805,543.

Ethanol (10 ml) and 2 M NaOH_{(aq)} (10 ml) were added to a solution of 1-Mn (14.3 mg, 21.9 μmol) in 6 ml of THF. The reaction was refluxed for 12 hours followed by neutralization with 3 M H₂SO_{4(aq)}. Purification first by sephadex LH-20 chromatography with ultrapure water followed by dialysis with ultrapure water gave the desired product as a red-brown solid in 85% yield. ESI MS found m/z=539.0 [M+], calcd for C₂₄H₁₂MnN₄O₈⁺, m/z=539.0. UV-vis (Hepes buffer, pH=7.0) λ_{abs}=325, 377, 397, 421, 465, 561, 592 nm.

Synthesis of [5,10,15,20-tetrakis(acetoxymethylcarboxy)porphyrinato]manganese(III) (Mn TAMP) and [5-carboxy-10,15,20-tris(acetoxymethylcarboxy)porphyrinato]manganese(III) (2-MnTriAMP)

1-MnTCP (23.2 mg, 38.3 μmol) was dissolved in ultrapure water (5 ml). 1 M HCl_{(aq)} was added dropwise until the pH reached 2.2 and precipitation of a red solid occurred. After cooling in an ice-water bath for 30 minutes the solid was filtered and the clear filtrate was discarded. Cold ultrapure water was used to wash the solid and drying under under reduced pressure resulted in protonated 1-MnTCP (12.3 mg, 22.8 μmol). The water wash contained salt and 1-MnTCP and was subjected to dialysis. Protonated 1-MnTCP (9.8 mg, 18.2 μmol) was suspended in DMF (2 ml). Under continuous stirring, 1,8-Diazabicyclo[5.4.0]undec-7-ene (DBU, 36 μL, 145 μmol, 8 eq.) was added dropwise. After 10 min. the first aliquot of acetoxymethyl bromide (6 μl, 61 μmol, 3.4 eq) was added dropwise. The reaction temperature was maintained at 55° C. for 30 hours. The progress of the reaction was monitored by TLC. After 6 hours a second aliquot of acetoxymethyl bromide (6 μl, 61 μmol, 3.4 eq) was added dropwise. After 24 hours a third aliquot of acetoxymethyl bromide (3 μl, 31 μmol, 1.7 eq) was added. Distillation of DMF at 60° C. under reduced pressure resulted in a crude dark oil. The crude material was dissolved in DCM and washed with water twice, then washed with brine twice. The organic layer was dried over sodium sulphate and filtered prior to concentration on a rotary evaporator. Purification by stepped gradient column chromatography (eluting with DCM to 10% MeOH in DCM) on silica gel gave 6.8 mg (50%) of compound 2-MnTriAMP as a red-brown solid and 5% of MnTetraAMP as a red-brown solid. Characterization of MnTAMP: ESI MS positive mode: found m/z=827.0885 [M⁺], calculated for C₃₆H₂₈MnN₄O₁₆⁺, m/z=827.0875 and characterization of 2-MnTriAMP: ESI MS found m/z=755.0678 [M⁺], calculated for C₃₃H₂₄MnN₄O₁₄, m/z=755.0664, IR (cm⁻¹) 1744.27 (C═O), 1555.34 (COO⁻).

### Cell Labeling, Viability, MRI

Human breast cancer cells MDA-MB-231 were grown at 37° C. with 5% CO₂ in 1640-RPMI medium supplemented with 10% fetal bovine serum and 0.5% penicillin streptomycin. Contrast agents were quantified for manganese content by flame AAS prior to cell labeling. MnTCP was dissolved in ultrapure water and MnTriAMP was dissolved in DMSO to give 17 mM stock solutions. The stock solutions were added to the medium with the cells resulting in 83 μM incubation of 2-MnTriAMP or 1-MnTCP for 2 hours. The medium was removed and the cells were washed with fresh medium 5× and then were harvested by washing with PBS followed by addition of 0.05% trypsin EDTA to detach the cells. Viability was assessed directly after labeling with trypan blue exclusion test and observation of cell morphology by phase microscopy. The results are listed in Table 1.

TABLE 1
Viability measurements for MDA-MB-231 breast cancer cell pellets:
directly after incubation and 3 days after incubation

| Day | Treatment | Viability (%) (±SEM) |
|---|---|---|
| 0 hours after | Control | 99 ± 0 |
| 2 h treatment | 0.5% DMSO only | 98 ± 2 |
| | 1-MnTCP | 98 ± 1 |
| | 2-MnTriAMP | 97 ± 2 |
| 3 days after | Control | Not measured |
| 2 h treatment | 0.5% DMSO only | 90 ± 7 |
| | 1-MnTCP | 80 ± 6 |
| | 2-MnTriAMP | 87 ± 6 |

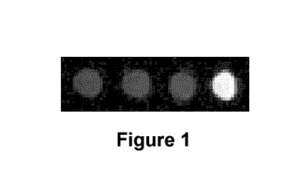

For MRI, cell pellets were formed by centrifugation at 440 g for 10 minutes. Subsequent MR was done with a T₁-weighted 2D spin-echo image: TR=100 ms, TE=14.163 ms, 3 mm slice thickness, 0.5×0.5 mm in-plane resolution (FIG. 1). The signal was analyzed to give the relaxivity of the cell pellets as seen in Table 2.

TABLE 2
T₁ and T₂ time measurements in MDA-
MB-231 breast cancer cell pellets

| | T₁ (ms) | T₂ (ms) |
|---|---|---|
| Control | 1201.6 ± 31.1 | 109.8 ± 9.8 |
| DMSO only | 1107.3 ± 26.6 | 108.6 ± 9.5 |
| Labeled with 1-MnTCP | 1072.1 ± 28.0 | 108.1 ± 9.3 |
| Labeled with 2-MnTriAMP in DMSO | 215.4 ± 12.1 | 66.7 ± 4.4 |

The intracellular contrast agent concentration was determined after cell labeling by centrifugation of a known number of cells followed by removal of the supernatant and digestion of the cells by addition of 1 M nitric acid (0.5 ml) and 30 min of heating at 70° C. Then 3 ml of ultrapure water was added to the digested cells, the mixture was filtered and then quantified by graphite furnace atomic absorption spectroscopy (GFAAS) shown in Table 3.

TABLE 3
GFAAS results of cell digests

| | Mn | Mn content per cell |
|---|---|---|
| Sample ID | (mM/cell) | relative to control |
| Control | 5.72E−15 | 1 |
| DMSO | 1.24E−14 | 2.17 |
| 1-MnTCP | 1.90E−13 | 33.22 |
| 2-MnTriAMP | 6.05E−13 | 105.83 |

The cell labeling showed significant uptake of the hydrophobic compound 2-MnTriAMP compared to the hydrophilic tetracarboxylic acid, 1-MnTCP as shown by the reduced relaxation times of the 2-MnTriAMP treated cells and the quantitative GFAAS data. The viability showed a statistically insignificant drop in healthy cells indicating lack of toxicity of the contrast agents after labeling. Therefore this compound shows great promise as a cell permeable and trappable positive contrast agent for cellular MRI applications.

This invention covers the structure, preparation and applications of a series of paramagnetic porphyrins designed as MRI T₁ contrast agent for cell-labeling and tracking. A selected compound from this series, 2-MnTriAMP was shown to be cell permeable with sufficient cellular uptake and retention in living cells, as well as a large T₁ contrast enhancement for high field MRI without affecting the cell viability. As such this invention has shown potential in efficient and safe applications for cell labeling and visualization with MRI in research and clinical settings.

### REFERENCES

1. Bulte, J. W. M., In Vivo MRI Cell Tracking: Clinical Studies. American Journal of Roentgenology 2009, 193 (2), 314-325.
2. Tsien Roger, Y., Fluorescent and Photochemical Probes of Dynamic Biochemical Signals inside Living Cells. In Fluorescent Chemosensors for Ion and Molecule Recognition, American Chemical Society: 1993; Vol. 538, pp 130-146.
3. de Silva, A. P.; Gunaratne, H. Q. N.; Gunnlaugsson, T.; Huxley, A. J. M.; McCoy, C. P.; Rademacher, J. T.; Rice, T. E., Signaling Recognition Events with Fluorescent Sensors and Switches. Chemical Reviews 1997, 97 (5), 1515-1566.
4. Bissell Richard, A.; de Silva, A. P.; Gunaratne, H. Q. N.; Lynch, P. L. M.; McCoy Colin, P.; Maguire Glenn, E. M.; Sandanayake, K. R. A. S., Fluorescent Photoinduced Electron-Transfer Sensors. In Fluorescent Chemosensors for Ion and Molecule Recognition, American Chemical Society: 1993; Vol. 538, pp 45-58.
5. Valeur, B.; Bourson, J.; Pouget, J., Ion Recognition Detected by Changes in Photoinduced Charge or Energy Transfer. In Fluorescent Chemosensors for Ion and Molecule Recognition, American Chemical Society: 1993; Vol. 538, pp 25-44.
6. Czarnik Anthony, W., Supramolecular Chemistry, Fluorescence, and Sensing. In Fluorescent Chemosensors for Ion and Molecule Recognition, American Chemical Society: 1993; Vol. 538, pp 1-9.
7. Giepmans, B. N. G.; Adams, S. R.; Ellisman, M. H.; Tsien, R. Y., The Fluorescent Toolbox for Assessing Protein Location and Function. Science 2006, 312 (5771), 217-224.
8. Ralph Weissleder, B. D. R., Alnawaz Rehemtulla, Sanjiv Sam Gambhir, Molecular Imaging. People's Medical Publishing House-USA: Shelton, Conn., USA, 2010.
9. Srinivas, M.; Heerschap, A.; Ahrens, E. T.; Figdor, C. G.; de Vries, I. J., (19)F MRI for quantitative in vivo cell tracking. Trends in biotechnology 2010, 28 (7), 363-70.
10. Muja, N.; Bulte, J. W. M., Magnetic resonance imaging of cells in experimental disease models. Progress in Nuclear Magnetic Resonance Spectroscopy 2009, 55 (1), 61-77.
11. Toso, C.; Vallee, J. P.; Morel, P.; Ris, F.; Demuylder-Mischler, S.; Lepetit-Coiffe, M.; Marangon, N.; Saudek, F.; James Shapiro, A. M.; Bosco, D.; Berney, T., Clinical Magnetic Resonance Imaging of Pancreatic Islet Grafts After Iron Nanoparticle Labeling. American Journal of Transplantation 2008, 8 (3), 701-706.
12. de Vries, I. J. M.; Lesterhuis, W. J.; Barentsz, J. O.; Verdijk, P.; van Krieken, J. H.; Boerman, O. C.; Oyen, W. J. G.; Bonenkamp, J. J.; Boezeman, J. B.; Adema, G. J.; Bulte, J. W. M.; Scheenen, T. W. J.; Punt, C. J. A.; Heerschap, A.; Figdor, C. G., Magnetic resonance tracking of dendritic cells in melanoma patients for monitoring of cellular therapy. Nat Biotech 2005, 23 (11), 1407-1413.
13. F., C.; C. M., D. M., Magnetic resonance tracking of magnetically labeled autologous bone marrow CD34+ cells transplanted into the spinal cord via lumbar puncture technique in patients with chronic spinal cord injury. Stem Cells and Development 2007, 16 (3), 461-466.
14. J., Z.; L., Z.; F., X., Tracking neural stem cells in patients with brain trauma. New England Journal of Medicine 2006, 355, 2376-2378.
15. Björklund, L. M.; Sánchez-Pernaute, R.; Chung, S.; Andersson, T.; Chen, I. Y. C.; McNaught, K. S. P.; Brownell, A.-L.; Jenkins, B. G.; Wahlestedt, C.; Kim, K.-S.; Isacson, O., Embryonic stem cells develop into functional dopaminergic neurons after transplantation in a Parkinson rat model. Proceedings of the National Academy of Sciences 2002, 99 (4), 2344-2349.
16. Ricordi, C.; Strom, T. B., Clinical islet transplantation: advances and immunological challenges. Nat Rev Immunol 2004, 4 (4), 259-268.
17. Heiser, A.; Coleman, D.; Dannull, J.; Yancey, D.; Maurice, M. A.; Lallas, C. D.; Dahm, P.; Niedzwiecki, D.; Gilboa, E.; Vieweg, J., Autologous dendritic cells transfected with prostate-specific antigen RNA stimulate CTL responses against metastatic prostate tumors. The Journal of clinical investigation 2002, 109 (3), 409-17.
18. Banchereau, J.; Steinman, R. M., Dendritic cells and the control of immunity. Nature 1998, 392 (6673), 245-252.
19. Nestle, F. O.; Banchereau, J.; Hart, D., Dendritic cells: On the move from bench to bedside. Nature medicine 2001, 7 (7), 761-5.
20. Orlic, D.; Kajstura, J.; Chimenti, S.; Jakoniuk, I.; Anderson, S. M.; Li, B.; Pickel, J.; McKay, R.; Nadal-Ginard, B.; Bodine, D. M.; Leri, A.; Anversa, P., Bone marrow cells regenerate infarcted myocardium. Nature 2001, 410 (6829), 701-5.
21. Chen, J.; Li, Y.; Wang, L.; Zhang, Z.; Lu, D.; Lu, M.; Chopp, M., Therapeutic Benefit of Intravenous Administration of Bone Marrow Stromal Cells After Cerebral Ischemia in Rats. Stroke 2001, 32 (4), 1005-1011.
22. Bulte, J. W.; Hoekstra, Y.; Kamman, R. L.; Magin, R. L.; Webb, A. G.; Briggs, R. W.; Go, K. G.; Hulstaert, C. E.; Miltenyi, S.; The, T. H.; et al., Specific MR imaging of human lymphocytes by monoclonal antibody-guided dextran-magnetite particles. Magnetic Resonance in Medicine: official journal of the Society of Magnetic Resonance in Medicine/Society of Magnetic Resonance in Medicine 1992, 25 (1), 148-57.
23. Bulte, J. W.; Douglas, T.; Witwer, B.; Zhang, S. C.; Strable, E.; Lewis, B. K.; Zywicke, H.; Miller, B.; van Gelderen, P.; Moskowitz, B. M.; Duncan, I. D.; Frank, J. A., Magnetodendrimers allow endosomal magnetic labeling and in vivo tracking of stem cells. Nature Biotechnology 2001, 19 (12), 1141-7.
24. Abragam, A., Principles of Nuclear Magnetism. Oxford University Press: Oxford, 1961.
25. Bernstein, M. A.; King, K. F.; Zhou, X. J., Handbook of MRI pulse sequences. Elsevier Academic Press: 2004.
26. Merbach A E, T. E., The Chemistry of Contrast Agents in Medical Magnetic Resonance Imaging. John Wiley & Sons, Ltd: Chichester, 2001; p 471.
27. Caravan, P.; Ellison, J. J.; McMurry, T. J.; Lauffer, R. B., Gadolinium(III) Chelates as MRI Contrast Agents: Structure, Dynamics, and Applications. Chemical Reviews 1999, 99 (9), 2293-2352.
28. Taupitz, M.; Schmitz, S.; Hamm, B., [Superparamagnetic iron oxide particles: current state and future development]. RoFo: Fortschritte auf dem Gebiete der Rontgenstrahlen and der Nuklearmedizin 2003, 175 (6), 752-65.
29. Bulte, J. W.; Kraitchman, D. L., Iron oxide MR contrast agents for molecular and cellular imaging. NMR Biomed 2004, 17 (7), 484-99.
30. Frank, J. A.; Zywicke, H.; Jordan, E. K.; Mitchell, J.; Lewis, B. K.; Miller, B.; Bryant, L. H., Jr.; Bulte, J. W., Magnetic intracellular labeling of mammalian cells by combining (FDA-approved) superparamagnetic iron oxide MR contrast agents and commonly used transfection agents. Academic radiology 2002, 9 Suppl 2, S484-7.
31. Shapiro, E. M.; Skrtic, S.; Sharer, K.; Hill, J. M.; Dunbar, C. E.; Koretsky, A. P., MRI detection of single particles for cellular imaging. Proceedings of the National Academy of Sciences of the United States of America 2004, 101 (30), 10901-10906.
32. Mani, V.; Briley-Saebo, K. C.; Itskovich, V. V.; Samber, D. D.; Fayad, Z. A., Gradient echo acquisition for superparamagnetic particles with positive contrast (GRASP): sequence characterization in membrane and glass superparamagnetic iron oxide phantoms at 1.5T and 3T. Magnetic resonance in medicine: official journal of the Society of Magnetic Resonance in Medicine/Society of Magnetic Resonance in Medicine 2006, 55 (1), 126-35.
33. Ahrens, E. T.; Flores, R.; Xu, H.; Morel, P. A., In vivo imaging platform for tracking immunotherapeutic cells. Nat Biotech 2005, 23 (8), 983-987.
34. Janjic, J. M.; Ahrens, E. T., Fluorine-containing nanoemulsions for MRI cell tracking. Wiley Interdisciplinary Reviews: Nanomedicine and Nanobiotechnology 2009, 1 (5), 492-501.
35. Li, W.-h.; Parigi, G.; Fragai, M.; Luchinat, C.; Meade, T. J., Mechanistic Studies of a Calcium-Dependent MRI Contrast Agent. Inorganic Chemistry 2002, 41 (15), 4018-4024.
36. Modo, M.; Cash, D.; Mellodew, K.; Williams, S. C. R.; Fraser, S. E.; Meade, T. J.; Price, J.; Hodges, H., Tracking Transplanted Stem Cell Migration Using Bifunctional, Contrast Agent-Enhanced, Magnetic Resonance Imaging. NeuroImage 2002, 17(2), 803-811.
37. Allen, M.; Meade, T., Synthesis and visualization of a membrane-permeable MRI contrast agent. J Biol Inorg Chem 2003, 8 (7), 746-750.
38. Allen, M. J.; MacRenaris, K. W.; Venkatasubramanian, P. N.; Meade, T. J., Cellular delivery of MRI contrast agents. Chemistry & biology 2004, 11 (3), 301-7.
39. Endres, P. J.; MacRenaris, K. W.; Vogt, S.; Meade, T. J., Cell-Permeable MR Contrast Agents with Increased Intracellular Retention. Bioconjugate chemistry 2008, 19 (10), 2049-2059.
40. Mohs, A. M.; Lu, Z.-R., Gadolinium(III)-based blood-pool contrast agents for magnetic resonance imaging: status and clinical potential. Expert Opinion on Drug Delivery 2007, 4 (2), 149-164.
41. Haenold, R.; Herrmann, K.-H.; Schmidt, S.; Reichenbach, J. R.; Schmidt, K.-F.; Löwel, S.; Witte, O. W.; Weih, F.; Kretz, A., Magnetic resonance imaging of the mouse visual pathway for in vivo studies of degeneration and regeneration in the CNS. NeuroImage 2012, 59 (1), 363-376.
42. Aoki, I.; Takahashi, Y.; Chuang, K.-H.; Silva, A. C.; Igarashi, T.; Tanaka, C.; Childs, R. W.; Koretsky, A. P., Cell labeling for magnetic resonance imaging with the T1 agent manganese chloride. NMR in Biomedicine 2006, 19 (1), 50-59.
43. Simmons, J. M.; Saad, Z. S.; Lizak, M. J.; Ortiz, M.; Koretsky, A. P.; Richmond, B. J., Mapping Prefrontal Circuits In Vivo with Manganese-Enhanced Magnetic Resonance Imaging in Monkeys. The Journal of Neuroscience 2008, 28 (30), 7637-7647.
44. Yang, Y.; An, J.; Wang, Y.; Luo, W.; Wang, W.; Mei, X.; Wu, S.; Chen, J., Intrastriatal manganese chloride exposure causes acute locomotor impairment as well as partial activation of substantia nigra GABAergic neurons. Environmental Toxicology and Pharmacology 2011, 31 (1), 171-178.
45. Chen, C. w.; Cohen, J. S.; Myers, C. E.; Sohn, M., Paramagnetic metalloporphyrins as potential contrast agents in NMR imaging. FEBS Letters 1984, 168 (1), 70-74.
46. Koenig, S. H.; Ill, R. D. B.; Spiller, M., The anomalous relaxivity of Mn(III)TPPS4 Magnetic Resonance in Medicine 1987, 4 (3), 252-260.
47. Lindsey, J. S.; Schreiman, I. C.; Hsu, H. C.; Kearney, P. C.; Marguerettaz, A. M., Rothemund and Adler-Longo reactions revisited: synthesis of tetraphenylporphyrins under equilibrium conditions. The Journal of Organic Chemistry 1987, 52 (5), 827-836.
48. Trova, M. P.; Gauuan, P. J. F.; Pechulis, A. D.; Bubb, S. M.; Bocckino, S. B.; Crapo, J. D.; Day, B. J., Superoxide dismutase mimetics. Part 2: synthesis and structure—Activity relationship of glyoxylate- and glyoxamide-Derived metalloporphyrins. Bioorganic and Medicinal Chemistry 2003, 11 (13), 2695-2707.

1. A compound in the form of a metallized paramagnetic tetrapyrollic contrast agent linked to a substituent, wherein the substituent is hydrolysable by an enzyme to form a relatively polar group.

2. The compound of claim 1, wherein the hydrolysable substituent contains a hydrolysable covalent linkage selected from the group consisting of ester (—C(O)O—), ether (—O—), amide (—C(O)NH—), alkylamide (—C(O)NR—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage.

3. The compound of claim 1, wherein the enzyme is an esterase.

4. The compound of claim 1 having the formula (B): 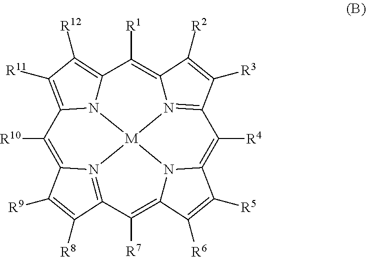
wherein M is a paramagnetic metal ion;
at least one of R¹ to R¹² is said hydrolysable substituent, and each of the remaining R¹ to R¹² is independently selected from the group consisting of:
hydrogen;
C₁-C₂₀ alkyl optionally substituted with up to 4 of any of hydroxyl, halogen, thiol, cyano, nitro, oxo (═O);
C₃-C₂₀ cycloalkyl optionally substituted with up to 4 of any of hydroxyl, halogen, thiol, cyano, nitro, oxo (═O);
C₃-C₂₀ heterocycloalkyl optionally substituted with up to 4 of any of hydroxyl, halogen, thiol, cyano, nitro, oxo (═O);
C₁-C₂₀ alkenyl optionally substituted with up to 4 of any of hydroxyl, halogen, thiol, cyano, nitro, oxo (═O);
C₃-C₂₀ cycloalkenyl optionally substituted with up to 4 of any of hydroxyl, halogen, thiol, cyano, nitro, oxo (═O);
C₃-C₂₀ heterocycloalkenyl optionally substituted with up to 4 of any of hydroxyl, halogen, thiol, cyano, nitro, oxo (═O);
C₆ to C₂₀ aryl optionally substituted with up to 4 of any of hydroxyl, halogen, thiol, cyano, nitro;
C₃ to C₂₀ heteroaryl optionally substituted with up to 4 of any of hydroxyl, halogen, thiol, cyano, nitro;
C₇ to C₂₀ arylalkyl optionally substituted with up to 4 of any of hydroxyl, halogen, thiol, cyano, nitro, oxo (═O);
C₄ to C₂₀ heteroarylalkyl optionally substituted with up to 4 of any of hydroxyl, halogen, thiol, cyano, nitro, oxo (═O);
C₂ to C₂₀ alkynyl optionally substituted with up to 4 of any of hydroxyl, halogen, thiol, cyano, nitro, oxo (═O);
C₁ to C₂₀ heteroalkyl optionally substituted with up to 4 of any of hydroxyl, halogen, thiol, cyano, nitro, oxo (═O);
C₂ to C₂₀ heteroalkenyl optionally substituted with up to 4 of any of hydroxyl, halogen, thiol, cyano, nitro, oxo (═O); and
C₂ to C₂₀ heteroalkynyl optionally substituted with up to 4 of any of hydroxyl, halogen, thiol, cyano, nitro, oxo (═O).

5. The compound of claim 4, wherein said at least one of R¹ to R¹² is selected from the group of substituents consisting of: 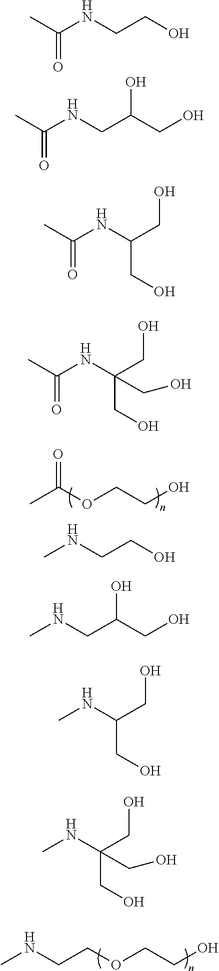
for n=1 to 20,
wherein at least one of the hydroxyl groups of each substituent is replaced by a linkage covalently linked to a protecting group selected from the group consisting of: ester (—C(O)O—), ether (—O—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage.

6. The compound of claim 4, wherein said at least one of R¹ to R¹² is selected from the group consisting of: 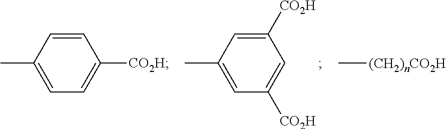
wherein the hydrogen atom of least one carboxyl group is substituted by a C₁-C₂₀ alkyl in which each hydrogen atom is optionally substituted with a halogen atom.

7. The compound of claim 4, wherein said at least one of R¹ to R¹² is: 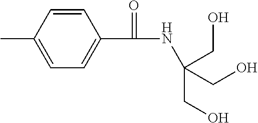 [SMILES: Cc1ccc(C(=O)NC(CO)(CO)CO)cc1]
wherein at least one of the hydroxyl groups is replaced by a linkage covalently linked to a protecting group selected from the group consisting of: ester (—C(O)O—), ether (—O—), phosphoryl (—OP(O)(OH)O—), alkylphosphoryl (—OP(O)(OR)O—), phosphonyl (—P(O)(OH)O—), alkylphosphonyl (—P(O)(OR)O—), sulfonyl (—S(O)(OH)O—), and alkylsulfonyl (—S(O)(OR)O—), wherein R is (C1-C20) alkyl group, branched or unbranched, and optionally substituted with one or more of halogen, and including salt forms of the phosphoryl, phosphonyl, sulfuryl and sulfonyl linkage.

8. The compound of claim 4, wherein a leaving group is covalently bonded to said linkage, the leaving group being selected from the group consisting of: 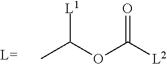
and L¹ and L² are independently selected from H and (C1-C20) alkyl, in particular:

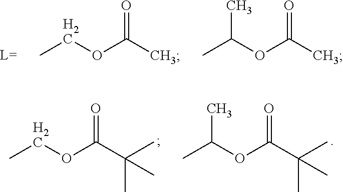

9. The compound of claim 1, the compound selected from the group consisting of: 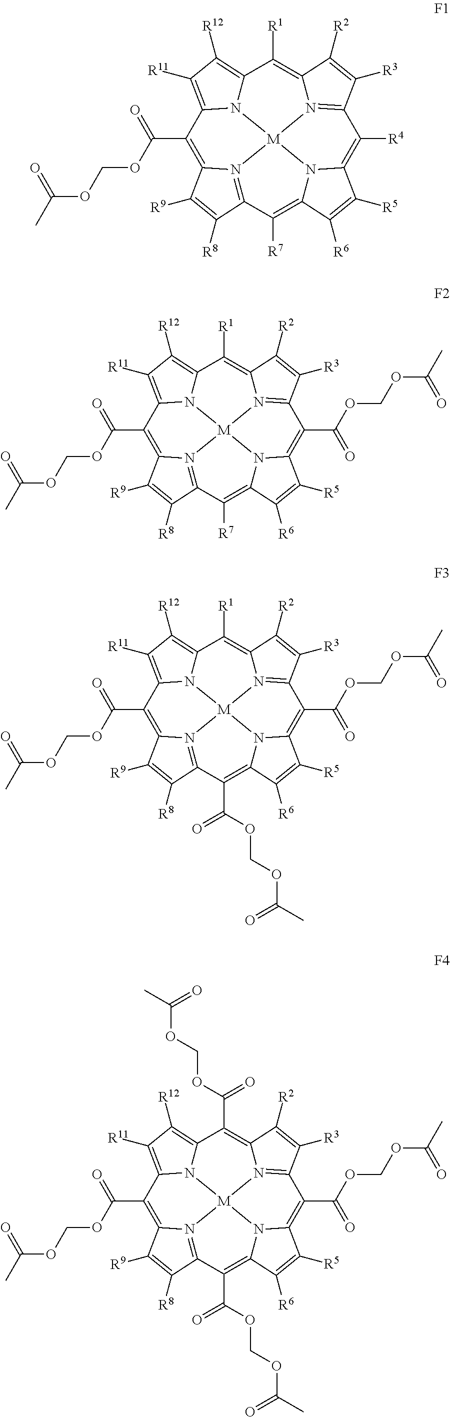
for R¹ to R¹² being defined as in claim 4.

10. The compound of claim 4, wherein R¹, R⁴, R⁷ and R¹¹ are independently selected from H and the following:

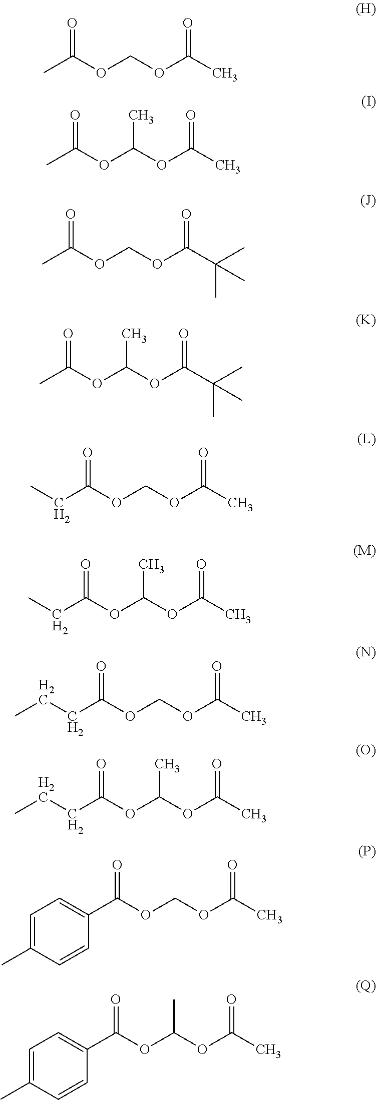

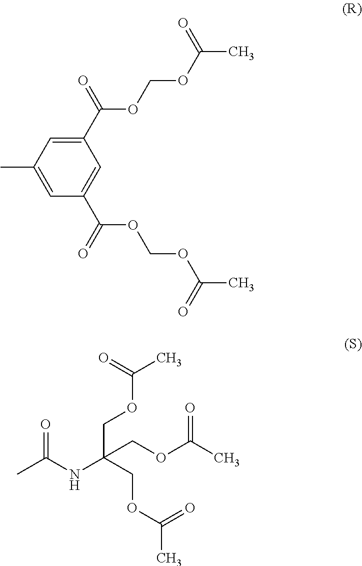

11. The compound of claim 10, selected from the group consisting of:

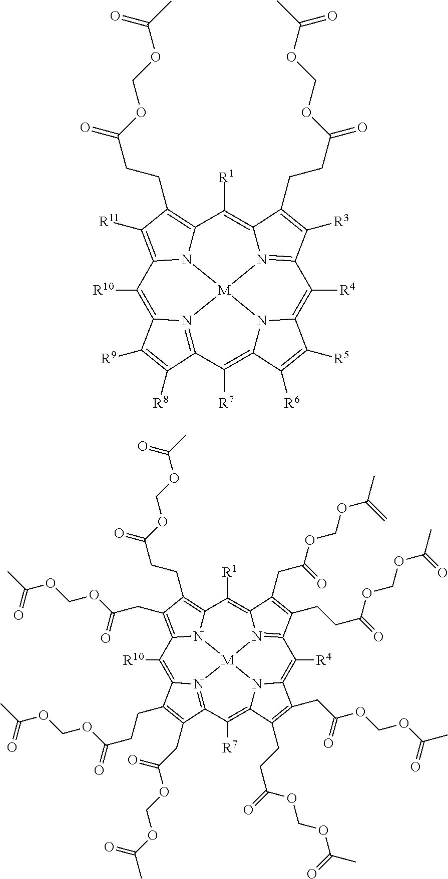

12. The compound of claim 10, selected from the group consisting of:

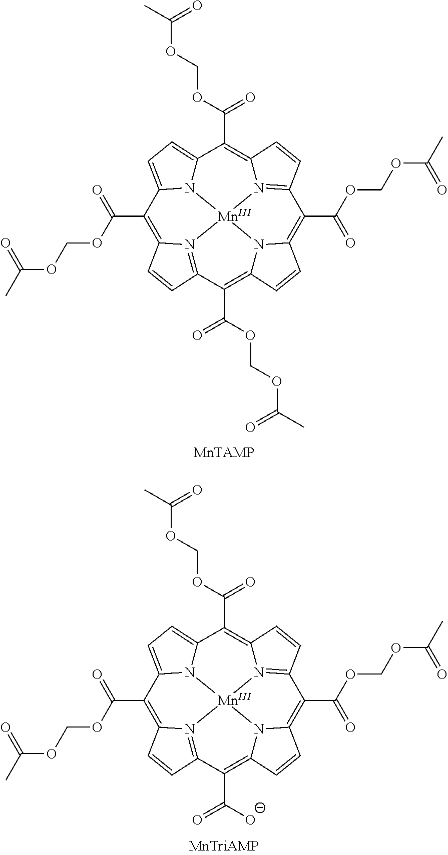

13. The compound according to claim 1, comprising a first and second porphyrin rings having the structure of respective formulas (P) and (P′): 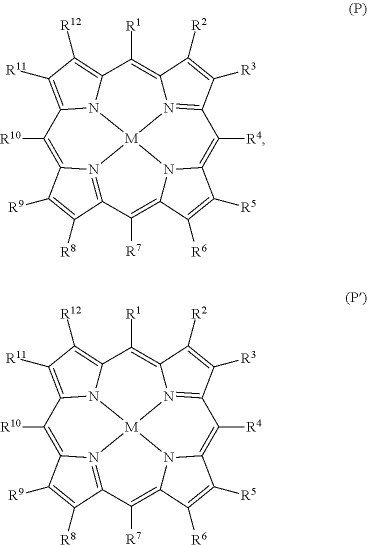
one of R¹ to R¹² of each of (P) and (P′) is independently a link to the other of (P) and (P′), the link being a covalent bond or the diradical of biphenyl (—C₆H₄—C₆H₄—), there being one, two, three or four porphyrin rings (P) linked to (P′);
at least one of the remaining R¹ to R¹² of each said porphyrin ring (P) is said hydrolysable substituent, said substituent being hydrophilic and having a protecting group covalently linked thereto, such that the covalent linkage of the protecting group is hydrolysable by the enzyme under physiologic conditions to produce an unprotected agent that is relatively hydrophilic with respect to the protected agent;
optionally, each said (P) linked to (P′) is linked to another porphyrin ring (P); and
each remaining R¹ to R¹² is selected from the group consisting of:
hydrogen;
C₁-C₂₀ alkyl optionally substituted with up to 4 of any of hydroxyl, halogen, thiol, cyano, nitro, oxo (═O);
C₃-C₂₀ cycloalkyl optionally substituted with up to 4 of any of hydroxyl, halogen, thiol, cyano, nitro, oxo (═O);
C₃-C₂₀ heterocycloalkyl optionally substituted with up to 4 of any of hydroxyl, halogen, thiol, cyano, nitro, oxo (═O);
C₁-C₂₀ alkenyl optionally substituted with up to 4 of any of hydroxyl, halogen, thiol, cyano, nitro, oxo (═O);
C₃-C₂₀ cycloalkenyl optionally substituted with up to 4 of any of hydroxyl, halogen, thiol, cyano, nitro, oxo (═O);
C₃-C₂₀ heterocycloalkenyl optionally substituted with up to 4 of any of hydroxyl, halogen, thiol, cyano, nitro, oxo (═O);
C₆ to C₂₀ aryl optionally substituted with up to 4 of any of hydroxyl, halogen, thiol, cyano, nitro;
C₃ to C₂₀ heteroaryl optionally substituted with up to 4 of any of hydroxyl, halogen, thiol, cyano, nitro;
C₇ to C₂₀ arylalkyl optionally substituted with up to 4 of any of hydroxyl, halogen, thiol, cyano, nitro, oxo (═O);
C₄ to C₂₀ heteroarylalkyl optionally substituted with up to 4 of any of hydroxyl, halogen, thiol, cyano, nitro, oxo (═O);
C₂ to C₂₀ alkynyl optionally substituted with up to 4 of any of hydroxyl, halogen, thiol, cyano, nitro, oxo (═O);
C₁ to C₂₀ heteroalkyl optionally substituted with up to 4 of any of hydroxyl, halogen, thiol, cyano, nitro, oxo (═O);
C₂ to C₂₀ heteroalkenyl optionally substituted with up to 4 of any of hydroxyl, halogen, thiol, cyano, nitro, oxo (═O); and
C₂ to C₂₀ heteroalkynyl optionally substituted with up to 4 of any of hydroxyl, halogen, thiol, cyano, nitro, oxo (═O).

14. The compound of claim 13, selected from the group consisting of:

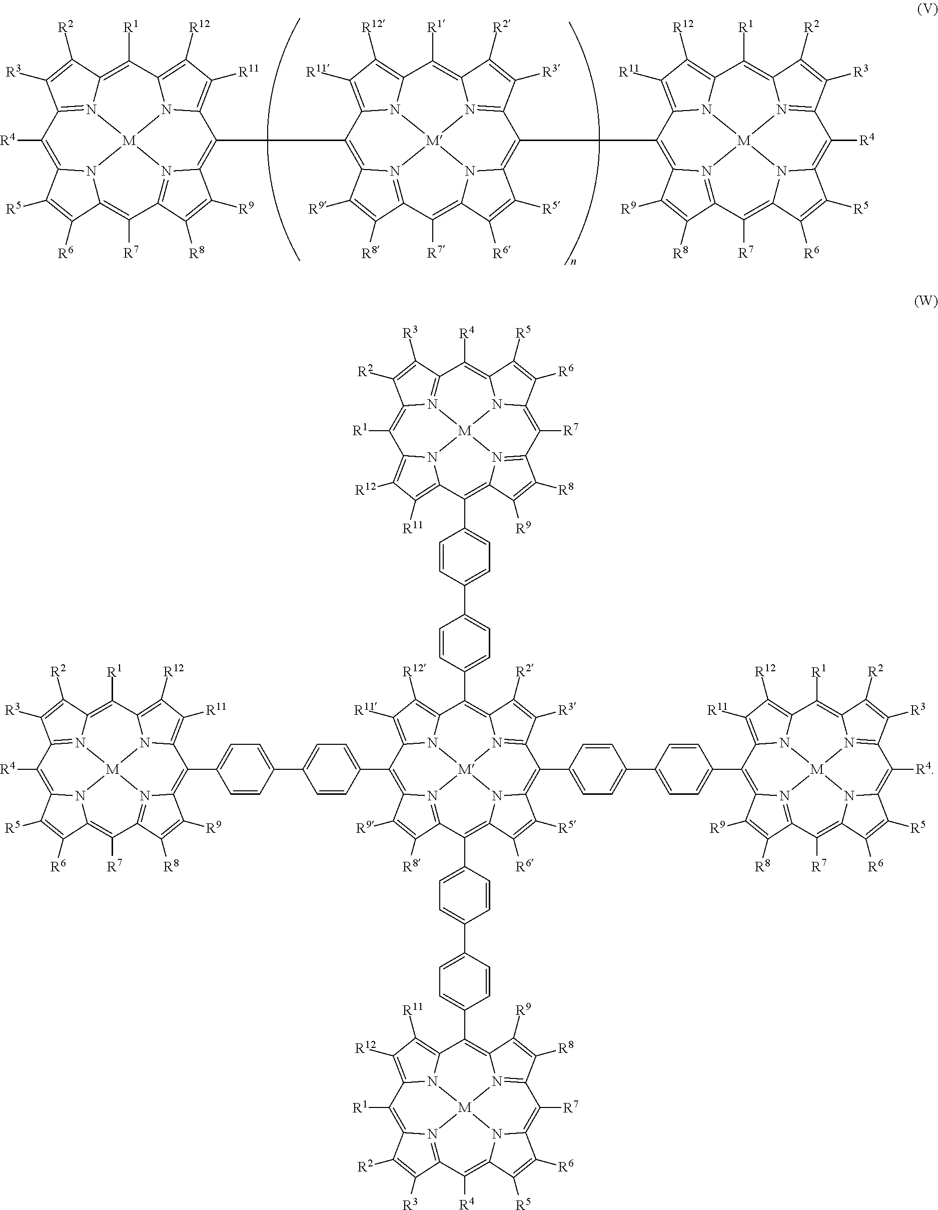

15. The compound of claim 1, wherein the paramagnetic metal is selected from the group consisting of Mn(II), Mn(III), Fe(II), Fe(III), Gd(III), Cu(I), Cu(II), Ni(II), Ni(I) and Ni(III), wherein the metal in each ring of a said compound may be the same or different.

16. The pharmaceutical composition of claim 23 suitable for administration as an imaging enhancing agent and the contrast agent is present in an amount sufficient to enhance a magnetic resonance image.

17. (canceled)

18. A method of enhancing an image of a cell, the method comprising:
exposing the cell to a contrast agent comprising a porphyrin ring covalently linked to a hydrophobic group by an ester linkage;
waiting a sufficient time permit the contrast agent to migrate across the cell membrane into the interior of the cell and for an esterase of the cell to cleave the hydrophobic group from the ring to generate relatively hydrophilic contrast agent in the interior of the cell; and
generating an image of the cell.

19. The method of claim 18, wherein said contrast agent further comprises a relatively hydrophilic group linking the porphyrin ring and said ester linkage such that the hydrophilicity of the agent increases upon said cleavage by the esterase.

20. The method of claim 17, 18 or 19, wherein said cell is a stem cell, immune cell, blood cell, neuron, or beta cell.

21-22. (canceled)

23. A pharmaceutical composition comprising a compound or salt thereof, as claimed in claim 1, and a pharmaceutically acceptable carrier, excipient or diluent, suitable for administration to a subject.
